(19) United States
(12) Patent Application Publication
(10) Pub. No.: US 2007/0292962 A1
(43) Pub. Date: Dec. 20, 2007

(54) Methods and compositions for genetic markers for autism

(75) Inventors: Margaret Pericak-Vance, Coral Gables, FL (US); John R. Gilbert, Palmetto Bay, FL (US); Michael L. Cuccaro, Durham, NC (US); John P. Hussman, Ellicott City, MD (US)

Correspondence Address: MYERS BIGEL SIBLEY & SAJOVEC, PO BOX 37428, RALEIGH, NC, 27627, US

(73) Assignee: Duke University

(21) Appl. No.: 11/786,077

(22) Filed: Apr. 10, 2007

Related U.S. Application Data
(60) Provisional application No. 60/790,703, filed on Apr. 10, 2006.

(51) Int. Cl. C12Q 1/68

(52) U.S. Cl. 436/94

(57) ABSTRACT

The present invention provides methods of identifying a subject having an increased risk of developing autistic disorder, comprising: a) correlating the presence of one or more genetic markers within a GABAR subunit gene with an increased risk of developing autistic disorder; and b) detecting the one or more genetic markers of step (a) in the subject, thereby identifying the subject as having an increased risk of developing autistic disorder. Also provided are methods of identifying effective treatment regimens for autistic disorder, based on correlation with genetic markers a GABAR subunit gene. The present invention further provides methods of diagnosing an autistic disorder in a subject, comprising detecting genetic markers correlated with a diagnosis of an autistic disorder.

### STATEMENT OF PRIORITY

The present application claims the benefit, under 35 U.S.C. § 119(e), of U.S. Provisional Application No. 60/790,703, filed Apr. 10, 2006, the entire contents of which are incorporated by reference herein.

### GOVERNMENT SUPPORT

The present invention was made, in part, with the support of grant numbers NS26630 from the National Institutes of Health and NS36768 from the National Institute of Neurological Disorders and Stroke. The United States Government has certain rights to this invention.

### FIELD OF THE INVENTION

The present invention provides methods and compositions directed to identification of genetic markers and their correlation with autistic disorder.

### BACKGROUND OF THE INVENTION

Autistic disorder (AD [MIM 209850]) is a neurodevelopmental disorder characterized by impairments in reciprocal social interaction and communication and the presence of restricted and repetitive patterns of interest or behavior. These impairments are apparent in the first three years of life and persist into adulthood. With the improved detection and recognition of autism that has resulted from a broadening of the diagnostic concept and systematic population approaches, a recent prevalence study reported that autistic disorder affects as many as 1 in 300 children in a US metropolitan area (Yeargin-Allsopp et al. 2003). The increase in prevalence has drawn significant attention from scientists and a rapid increase in the level of interest in the etiology of autism has been seen in the past decade (Fombonne 1999; Fombonne 2003a).

Autism has turned out to be one of the most heritable complex genetic disorders in psychiatry. A strong genetic component in autism is indicated by an increased concordance rate in monozygotic (60% and 91% for the narrow and broader phenotypes respectively) versus dizygotic twins (0 and 10% for the narrow and broader phenotypes respectively) (Steffenburg et al. 1989; Bailey et al. 1995) and a 75-fold greater risk to siblings of idiopathic cases in comparison to the prevalence in the general population (Bolton et al. 1994). Collectively, these studies suggest that autistic disorder involves multiple variants in multiple unlinked loci interacting to cause the autism phenotype. In addition to genetic risk assessment studies, both direct (chromosomal methods, linkage and association studies) and indirect mapping approaches (the characterization of disorders that share some of the symptoms of autism such as Rett or fragile X syndrome) have been applied to identify autism susceptibility genes. These studies also yield convincing evidence for the multi-genic inheritance and locus or allelic heterogeneity in autism.

There are two approaches to identifying genetic contributors to disease. The first is a genome wide search in which linkage or association analysis is used to identify regions of the genome that may contain autism susceptibility genes. The second is the candidate gene approach, which investigates a specific gene or genes for involvement in autism risk. In the candidate gene approach, genes are chosen for study based on either what is known about the gene's function, its location (for example in a recognized linkage peak), or a combination of both. Several candidates are hypothesized to be involved in autism; however no single candidate gene has consistently emerged as involved in autism risk.

Over 10 genome-wide autism screens have been performed in attempts to identify the genetic basis of autism (International Molecular Genetic Study of Autism Consortium 1998; International Molecular Genetic Study of Autism Consortium 2001; Collaborative Linkage Study of Autism 2001; Liu et al. 2001; Meyers et al. 1998; Shao et al. 2002a; Risch et al. 1999; Auranen et al. 2002; Philippe et al. 1999; Yonan et al. 2003). Results from these various screens indicate potential susceptibility genes spread across the entire genome. Estimates of the number of genes involved in autism range from 3 to 10 (Pickles et al. 1995; Folstein et al. 2001) to 15 or more (Risch et al. 1999) to 100 loci (Pritchard 2001). Numerous association studies on the candidate genes have been conducted based on both location in a linkage peak or potential function, but no single gene has been consistently replicated across studies. One explanation for the low efficiency of association studies is that there are many contributing genetic and environmental factors in autism. Moreover, multiple interacting genes may be the main causative determinants of autism (Muhle et al. 2004; Veenstra-VanderWeele et al. 2004). With only a modest sample size, a small to moderate locus effect is not easily detected. Therefore, tests for joint effects may be more successful in the search for autism susceptibility genes.

One candidate pathway that is hypothesized to be involved in autism is the GABAergic system. Hussman (2001) suggested that autism is the result of an imbalance of the excitatory glutamatergic and inhibitory GABAergic pathways, resulting in over-stimulation in the brain and inability to filter out excess stimuli from environmental and intrinsic sources. Multiple lines of evidence support this theory. First, histological, biochemical, and molecular approaches have demonstrated altered levels and distribution of GABA (gamma-aminobutyric acid) and GABA receptors in peripheral blood and plasma, as well as in the brain, including decreased GABA-A receptors and benzodiazepine binding sites in the hippocampal formation (Rolf et al. 1993; Dhossche et al. 2002; Blatt et al. 2001). There are also reported alterations in GABAergic neurons, as demonstrated by the increased packing density of GABAergic interneurons in the CA3 and CA1 subfields, and by the decreased numbers and reduced size of cerebellar GABAergic Purkinje cells (Fatemi et al. 2002; Bauman et al. 2005). Duplications, isodicentric chromosomes, linkage, and association that include the three clustered GABA receptor subunits GABRB3, GABRA5, and GABRG3 on chromosome 15q have been associated with autism, as well (Buxbaum et al. 2005; Buxbaum et al. 2002; Shao et al. 2002b). Lastly, mutations have been reported in multiple GABA receptor genes in families with epilepsy (Macdonald et al. 2004). Given the high co-morbidity of autism with epilepsy and seizures, these data suggest that a similar molecular etiology could exist between the disorders.

Signaling in the GABAergic system is mediated by receptors for the neurotransmitter GABA. There are 19 known GABA receptor subunits arranged in clusters throughout the genome. Functional pentamers formed by various combinations of these subunits results in receptors of varying properties and sensitivities. The amounts and functional capabilities of individual receptor subunits that form a specific pentamer can affect the amount and quality of signaling in different parts of the brain.

### SUMMARY OF THE INVENTION

The present invention provides a method of identifying a subject having an increased risk of developing an autistic disorder, comprising detecting in the subject one or more genetic markers within a gamma-aminobutyric acid receptor (GABAR) subunit gene correlated with an increased risk of developing an autistic disorder.

In a further embodiment, the present invention provides a method of identifying a subject having an increased risk of developing an autistic disorder, comprising: a) correlating the presence of one or more genetic markers within a GABAR subunit gene with an increased risk of developing autistic disorder; and b) detecting the one or more genetic markers of step (a) in the subject, thereby identifying the subject as having an increased risk of developing autistic disorder.

Also provided is a method of correlating a genetic marker within a GABAR subunit gene with an increased risk of developing an autistic disorder, comprising: a) detecting in a subject with an autistic disorder the presence of one or more genetic markers within the GABAR subunit gene; and b) correlating the presence of the one or more genetic markers of step (a) with the autistic disorder in the subject.

Additionally, provided herein is a method of diagnosing an autistic disorder in a subject, comprising detecting in the subject one or more genetic markers correlated with a diagnosis of an autistic disorder.

Further provided is a method of diagnosing an autistic disorder in a subject, comprising: a) correlating the presence of one or more genetic markers within a GABAR subunit gene with a diagnosis of an autistic disorder; and b) detecting the one or more genetic markers of step (a) in the subject, thereby diagnosing an autistic disorder in the subject.

In yet additional embodiments, the present invention provides a method of correlating a genetic marker within a GABAR subunit gene with a diagnosis of an autistic disorder, comprising: a) detecting in a subject diagnosed with an autistic disorder the presence of one or more genetic markers within the GABAR subunit gene; and b) correlating the presence of the one or more genetic markers of step (a) with a diagnosis of an autistic disorder in a subject.

The present invention also provides a method of identifying an effective treatment regimen for a subject with an autistic disorder, comprising detecting one or more genetic markers within a GABAR subunit gene in the subject that is correlated with an effective treatment regimen for an autistic disorder.

In addition, the present invention provides a method of identifying an effective treatment regimen for a subject with an autistic disorder, comprising: a) correlating the presence of one or more genetic markers within a GABAR subunit gene in a test subject with an autistic disorder for whom an effective treatment regimen has been identified; and b) detecting the one or more markers of step (a) in the subject, thereby identifying an effective treatment regimen for the subject.

Also provided is a method of correlating a genetic marker within a GABAR subunit gene with an effective treatment regimen for autistic disorder, comprising: a) detecting in a subject with an autistic disorder and for whom an effective treatment regimen has been identified, the presence of one or more genetic markers within a GABAR subunit gene; and b) correlating the presence of the one or more genetic markers of step (a) with an effective treatment regimen for an autistic disorder.

### DETAILED DESCRIPTION OF THE INVENTION

Several lines of research indicate that there are abnormalities in the GABAergic system that may lead to developmental changes similar to those observed in autism. The evidence implicates GABA receptor (GABAR) subunit genes as functional candidates for autism (Blatt et al. 2001; Aldred et al. 2003; Hussman 2001). GABA (Hahn et al. 2003; Moore 2003) acts on the GABAR complex, a heteromeric structure, and mediates synaptic inhibition in the adult brain. During development, GABA also acts as an excitatory neurotransmitter due to the high intracellular chloride concentration in immature neurons (Jentsch et al. 2002). Eight GABA classes (α, β, δ, ε, γ, π, and ρ) and 18 receptor subunit genes have been characterized in mammals. In addition to providing binding sites for GABA, the GABAR contains sites for several therapeutic agents and drugs, including benzodiazepines, barbiturates, anesthetics, and alcohols. Binding studies using labeled ligands in children indicate that GABAR density is greater early in life and then dramatically decreases to adult levels (Chugani et al. 2001). Subunit composition varies developmentally and across brain structure. It is notable that the studies found a significant decrease in GABAR density in autism (Blatt et al. 2001) and an elevated plasma GABA level in autistic youngsters (Dhossche et al. 2002).

The most promising region identified by autism association studies is on chromosome 15q12, which harbors a set of 3 GABAR subunit genes (Martin et al. 2000a; Wolpert et al. 2000; Boyar et al. 2001; Menold et al. 2001; Buxbaum et al. 2002; Cook, Jr. et al. 1998). Chromosome 15q11-q13 duplications and deletions have also been documented in children with autism (Pujana et al. 2002; Bundey et al. 1994; Smith et al. 2000). In addition, several groups have identified this region as interesting through linkage studies (Philippe et al. 1999; Liu et al. 2001). Follow-up fine mapping narrowed this 15q region to the GABRB3 gene by use of a phenotypic subtype defined by a high degree of insistence on sameness (Shao et al. 2003). All of these findings from direct or indirect mapping studies strongly suggest that the GABAR subunit genes may play an important role in the etiology of autism both independently and interactively.

Epistasis or gene-gene interaction has been widely accepted as an important attributor to the complexity of mapping complex disease genes (Moore 2003). The failure to replicate some single locus results might be due to an underlying genetic architecture in which gene-gene interactions are the norm rather than the exception (Moore and Williams 2002). Thus, genetic studies that ignore epistasis or gene-gene interactions are only likely to reveal part of the genetic architecture. Although the term “epistasis” was initially used by William Bateson early in the 20^{th} century to describe the reason for distortions of mendelian segregation ratios and later defined by Fisher as deviations from additivity in a linear statistical model (Moore 2005), the methodology in testing for epistasis or gene-gene interaction is still in its infancy.

The available methods have been thoroughly reviewed recently (Thornton-Wells et al. 2004). In general, a lack of powerful statistical methods and large sample sizes limit the identification and characterization of gene-gene interactions (Moore and Williams 2002). The main issues confronted by traditional methods such as logistic regression are insufficient power and inflexibility to detect high-order gene-gene interactions. Several newly developed methods such as multi-locus geno-PDT (Martin et al. 2003a) and the multifactor dimensionality reduction (MDR) method (Ritchie et al. 2001) improve the ability to identify the high-order gene-gene interaction with relatively small sample sizes. However, they have difficulties distinguishing true interactive effects from joint effects. With the data-driven analytic methods that are continuously in development to examine complex genetic interactions, it has become increasingly important to stress model validation in order to ensure that significant effects represent true relationships rather than chance findings (Coffey et al. 2004). Thus, a multi-analytic approach to analysis of gene-gene interactions was proposed (Ashley-Koch et al. 2004), which searches for consistency of results and preponderance of evidence to draw the most useful conclusions. In the present invention, this new paradigm was applied to determine the contribution of the GABAR subunit genes to the etiology of autism independently and/or through complex interactions between subunit genes.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

As used herein, “a,” “an” or “the” can mean one or more than one. For example, “a” cell can mean a single cell or a multiplicity of cells.

As used herein, “and/or” refers to and encompasses any and all possible combinations of one or more of the associated listed items, as well as the lack of combinations when interpreted in the alternative (“or”).

Further, the term “about,” as used herein when referring to a measurable value such as an amount of a compound or agent of this invention, dose, time, temperature, and the like, is meant to encompass variations of ±20%, ±10%, ±5%, ±1%, +0.5%, or even ±0.1% of the specified amount.

As used herein, the term “autistic disorder” means a neurodevelopmental disorder characterized by impairments in reciprocal social interaction and communication and the presence of restricted and repetitive patterns of interest or behavior. Autistic disorder is one of a group of disorders called Pervasive Development Disorders (PDD). See also Diagnostic and Statistical Manual of Mental Disorders, published by the American Psychiatric Association (IV-TR, 2000).

Also as used herein, “linked” describes a region of a chromosome that is shared more frequently in family members affected by a particular disease or disorder, than would be expected or observed by chance, thereby indicating that the gene or genes or other identified marker(s) within the linked chromosome region contain or are associated with an allele that is correlated with the presence of, or increased or decreased risk of the disease or disorder. Once linkage is established, association studies (linkage disequilibrium) can be used to narrow the region of interest or to identify the marker correlated with the disease or disorder.

The term “genetic marker” as used herein refers to a region of a nucleotide sequence (e.g., in a chromosome) that is subject to variability (i.e., the region can be polymorphic for a variety of alleles). For example, a single nucleotide polymorphism (SNP) in a nucleotide sequence is a genetic marker that is polymorphic for two alleles. Other examples of genetic markers of this invention can include but are not limited to microsatellites, restriction fragment length polymorphisms (RFLPs), repeats (i.e., duplications), insertions, deletions, etc.

A subject of this invention is any animal that is susceptible to cardiovascular disease as defined herein and can include mammals, birds and reptiles. Examples of subjects of this invention can include, but are not limited to, humans, non-human primates, dogs, cats, horses, cows, goats, guinea pigs, mice, rats and rabbits, as well as any other domestic, commercially or clinically valuable animal including animal models of autistic disorder.

As used herein, “nucleic acids” encompass both RNA and DNA, including cDNA, genomic DNA, mRNA, synthetic (e.g., chemically synthesized) DNA and chimeras of RNA and DNA. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

The term “isolated” can refer to a nucleic acid or polypeptide that is substantially free of cellular material, viral material, or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

More specifically, an “isolated nucleic acid” is a DNA or RNA that is not immediately contiguous with nucleotide sequences with which it is immediately contiguous (one on the 5′ end and one on the 3′ end) in the naturally occurring genome of the organism from which it is derived. In other embodiments, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “oligonucleotide” refers to a nucleic acid sequence of at least about six nucleotides to about 100 nucleotides, for example, about 15 to 30 nucleotides, or about 20 to 25 nucleotides, which can be used, for example, as a primer in a PCR amplification or as a probe in a hybridization assay or in a microarray. Oligonucleotides can be natural or synthetic, e.g., DNA, RNA, modified backbones, etc.

The present invention is based in part on the inventor's discovery of a correlation between genetic markers in the gamma-aminobutyric acid receptor (GABAR) subunit genes. Thus, the present invention provides a method of identifying a subject having an increased risk of developing an autistic disorder, comprising detecting in the subject one or more genetic markers within a GABAR subunit gene correlated with an increased risk of developing an autistic disorder.

In further embodiments, the present invention provides a method of identifying a subject having an increased risk of developing an autistic disorder, comprising: a) correlating the presence of one or more genetic markers within a GABAR subunit gene with an increased risk of developing autistic disorder; and b) detecting the one or more genetic markers of step (a) in the subject, thereby identifying the subject as having an increased risk of developing autistic disorder.

Also provided is a method of correlating a genetic marker within a GABAR subunit gene with an increased risk of developing an autistic disorder, comprising: a) detecting in a subject with an autistic disorder the presence of one or more genetic markers within the GABAR subunit gene; and b) correlating the presence of the one or more genetic markers of step (a) with the autistic disorder in the subject.

Additionally provided herein is a method of diagnosing an autistic disorder in a subject, comprising detecting in the subject one or more genetic markers correlated with a diagnosis of an autistic disorder.

Further provided is a method of diagnosing an autistic disorder in a subject, comprising: a) correlating the presence of one or more genetic markers within a GABAR subunit gene with a diagnosis of an autistic disorder; and b) detecting the one or more genetic markers of step (a) in the subject, thereby diagnosing an autistic disorder in the subject.

In yet additional embodiments, the present invention provides a method of correlating a genetic marker within a GABAR subunit gene with a diagnosis of an autistic disorder, comprising: a) detecting in a subject diagnosed with an autistic disorder the presence of one or more genetic markers within the GABAR subunit gene; and b) correlating the presence of the one or more genetic markers of step (a) with a diagnosis of an autistic disorder in a subject.

In the methods described herein, the detection of a genetic marker in a subject can be carried out according to methods well known in the art. For example DNA is obtained from any suitable sample from the subject that will contain DNA and the DNA is then prepared and analyzed according to well-established protocols for the presence of genetic markers according to the methods of this invention. In some embodiments, analysis of the DNA can be carried out by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA)). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, or by electrophoresis. Thus, the present invention can further provide oligonucleotides for use as primers and/or probes for detecting and/or identifying genetic markers according to the methods of this invention.

The genetic markers of this invention are correlated with an autistic disorder as described herein according to methods well known in the art and as disclosed in the Examples provided herein for correlating genetic markers with various phenotypic traits, including disease states, disorders and pathological conditions and levels of risk associated with developing a disease, disorder or pathological condition. In general, identifying such correlation involves conducting analyses that establish a statistically significant association- and/or a statistically significant correlation between the presence of a genetic marker or a combination of markers and the phenotypic trait in the subject. An analysis that identifies a statistical association (e.g., a significant association) between the marker or combination of markers and the phenotype establishes a correlation between the presence of the marker or combination of markers in a subject and the particular phenotype being analyzed.

The correlation can involve one or more than one genetic marker of this invention (e.g., two, three, four, five, or more) in any combination. In some embodiments of this invention, the genetic markers are located in the gamma-aminobutyric acid receptor, alpha-4 (GABRA4) gene. In other embodiments, the genetic markers are located in the gamma-aminobutyric acid receptor, alpha-2 (GABRA2) gene. In further embodiments, the genetic markers are located in the gamma-aminobutyric acid receptor, beta-1 (GABRB1) gene. In additional embodiments, the genetic markers are located in the gamma-aminobutyric acid receptor, beta-2 (GABRB2) gene. In yet further embodiments, the genetic markers are located in the gamma-aminobutyric acid receptor, beta-3 (GABRB3) gene. In other embodiments of this invention, the genetic markers are located in the gamma-aminobutyric acid receptor, pi (GABRP) gene. In still other embodiments, the genetic markers are located in the gamma-aminobutyric acid receptor, rho-2 (GABRR2) gene. In further embodiments, genetic markers are located in the gamma-aminobutyric acid receptor, gamma-1 (GABRG1) gene. In still further embodiments, genetic markers are located in the gamma-aminobutyric acid receptor, gamma-3 (GABRG3) gene.

The genetic markers of this invention can be used individually or in combination. Thus, in some embodiments, the methods of this invention can include correlations between genetic markers located in the GABRA4 gene in combination with genetic markers located in other GABAR subunit genes and autistic disorder as described herein. For example, the genetic markers of this invention, such as those of the GABRA4 gene, can be combined with the genetic markers in the GABRB1 gene in the methods of this invention and in establishing correlations between genetic markers and various aspects of autistic disorder as described herein.

The genetic markers of the present invention are single nucleotide polymorphisms (SNP). Exemplary single nucleotide polymorphisms include but are not limited to T for G, T for A, C for A, C for T, A for G, A for C, A for T, G for A and G for T substitutions.

In some embodiments of the present invention, the single nucleotide polymorphism within the GABRA4 gene is selected from the group consisting of rs1912960, rs2280073, rs17599165, rs17599416, rs7660336, rs16859788, and any combination thereof. In other embodiments of the present invention, the single nucleotide polymorphism within the GABRB1 gene is selected from the group consisting of hcv2119841, rs2351299, rs4482737, rs383230, RS3114084, and any combination thereof. In yet other embodiments, the single nucleotide polymorphism within the GABRB2 gene is selected from the group consisting of RS2617503, RS12187676, and any combination thereof. In further embodiments, the single nucleotide polymorphism within the GABRB3 gene is RS1426217. In additional embodiments, the single nucleotide polymorphism within the GABRP gene is rs1862242. In some embodiments, the single nucleotide polymorphism within the GABRA2 gene is HCV8262334. In still other embodiments, the single nucleotide polymorphism within the GABRR2 gene is HCV9866022, RS2148174, RS2822117, and any combination thereof. In a further embodiment, the single nucleotide polymorphism within the GABRG1 gene is RS2350439. In a still further embodiment, the single nucleotide polymorphism within the GABRG3 gene is RS208129.

The present invention also provides a method wherein the genetic marker is a combination of the single nucleotide polymorphisms, or haplotypes, that is correlated with an aspect of autistic disorder as described herein. Thus, for example, haplotypes correlated with increased risk of autistic disorder or with a diagnosis of autistic disorder include rs1912960 within the GABRA4 gene and the single nucleotide polymorphism rs2351299 within the GABRB1 gene. Another embodiment provides a method wherein the genetic marker is a combination of the single nucleotide polymorphism rs2280073 within the GABRA4 gene and the single nucleotide polymorphism hcv2119841 within the GABRB1 gene. Also provided herein is a method wherein the genetic marker is a combination of the single nucleotide polymorphism rs2280073 within the GABRA4 gene and the single nucleotide polymorphism rs1862242 within the GABRP gene. Further provided is a method wherein the genetic marker is a combination of the single nucleotide polymorphism rs17599416 within the GABRA4 gene and the single nucleotide polymorphism rs2351299 within the GABRB1 gene. An additional embodiment of the present invention provides a method wherein the genetic marker is a combination of the single nucleotide polymorphism rs1912960 within the GABRA4 gene, the single nucleotide polymorphism rs2351299 within the GABRB1 gene and the single nucleotide polymorphism rs7660336 within the GABRA4 gene. Further provided herein is a method wherein the genetic marker is a combination of the single nucleotide polymorphism rs1912960 within the GABRA4 gene, the single nucleotide polymorphism rs2351299 within the GABRB1 gene and the single nucleotide polymorphism rs17599165 within the GABRA4 gene. Further embodiments provide a method wherein the genetic marker is a combination of the single nucleotide polymorphism rs1912960 within the GABRA4 gene, the single nucleotide polymorphism rs2351299 within the GABRB1 gene and the single nucleotide polymorphism rs17599416 within the GABRA4 gene. A method is also provided wherein the genetic marker is a combination of the single nucleotide polymorphism rs7660336 within the GABRA4 gene, the single nucleotide polymorphism rs2351299 within the GABRB1 gene and the single nucleotide polymorphism rs17599416 within the GABRA4 gene. Further provided is a method wherein the genetic marker is a combination of the single nucleotide polymorphism RS1912960 within the GABRA4 gene, the single nucleotide polymorphism RS3114084 within the GABRB1 gene and the single nucleotide polymorphism RS2350439 within the GABRG1 gene. The present invention also provides a method wherein the genetic marker is a combination of the single nucleotide polymorphisms RS282117 and RS2148174 within the GABRA4 gene, and the single nucleotide polymorphism RS208129 within the GABRG3 gene.

The present invention also provides a method of identifying an effective treatment regimen for a subject with an autistic disorder, comprising detecting one or more genetic markers within a GABAR subunit gene in the subject correlated with an effective treatment regimen for an autistic disorder.

In addition, the present invention provides a method of identifying an effective treatment regimen for a subject with an autistic disorder, comprising: a) correlating the presence of one or more genetic markers within a GABAR subunit gene in a test subject with an autistic disorder for whom an effective treatment regimen has been identified; and b) detecting the one or more markers of step (a) in the subject, thereby identifying an effective treatment regimen for the subject.

Also provided is a method of correlating a genetic marker within a GABAR subunit gene with an effective treatment regimen for autistic disorder, comprising: a) detecting in a subject with an autistic disorder and for whom an effective treatment regimen has been identified, the presence of one or more genetic markers within a GABAR subunit gene; and b) correlating the presence of the one or more genetic markers of step (a) with an effective treatment regimen for an autistic disorder.

Patients who respond well to particular treatment protocols can be analyzed for specific genetic markers and a correlation can be established according to the methods provided herein. Alternatively, patients who respond poorly to a particular treatment regimen can also be analyzed for particular genetic markers correlated with the poor response. Then, a subject who is a candidate for treatment for an autistic disorder can be assessed for the presence of the appropriate genetic markers and the most appropriate treatment regimen can be provided.

In some embodiments, the methods of correlating genetic markers with treatment regimens can be carried out using a computer database. Thus the present invention provides a computer-assisted method of identifying a proposed treatment for autistic disorder. The method involves the steps of (a) storing a database of biological data for a plurality of patients, the biological data that is being stored including for each of said plurality of patients (i) a treatment type, (ii) at least one genetic marker associated with autistic disorder and (iii) at least one disease progression measure for autistic disorder from which treatment efficacy can be determined; and then (b) querying the database to determine the dependence on said genetic marker of the effectiveness of a treatment type in treating autistic disorder, to thereby identify a proposed treatment as an effective treatment for a subject carrying a genetic marker correlated with autistic disorder.

In one embodiment, treatment information for a patient is entered into the database (through any suitable means such as a window or text interface), genetic marker information for that patient is entered into the database, and disease progression information is entered into the database. These steps are then repeated until the desired number of patients has been entered into the database. The database can then be queried to determine whether a particular treatment is effective for patients carrying a particular marker, not effective for patients carrying a particular marker, etc. Such querying can be carried out prospectively or retrospectively on the database by any suitable means, but is generally done by statistical analysis in accordance with known techniques, as described herein.

The present invention is more particularly described in the following examples that are intended as illustrative only since numerous modifications and variations therein will be apparent to those skilled in the art.

### EXAMPLES

### Example 1

### Family Ascertainment

A standard ascertainment protocol was conducted by the clinical groups at the Duke Center for Human Genetics and WS Hall Psychiatric Institute. Both sites recruited, enrolled, and sampled individuals with autism and family members per study protocols approved by their respective Institutional Review Boards (IRBs). Participating families were ascertained using clinical referrals and active recruitment through lay organizations providing services to families with autism. After a full description of the study was given to the families, written informed consent was obtained from parents as well as from children who were able to give informed consent. For the current study, the total number of Caucasian families is 470, of which 266 were multiplex (more than one affected individual sampled) and 204 were trios (only one affected individual sampled). The Collaborative Autism Team (CAT) from the Duke Center for Human Genetics and the WS Hall Psychiatric Institute contributed 246 families, while 224 families were from the Autism Genetic Resource Exchange (AGRE). Probands for the study consisted of individuals between the ages of 3 and 21 who were clinically diagnosed with autism using DSM-IV criteria. A consistent set of diagnostic criteria was applied to all families. Qualified individuals and families were those who met best estimate clinical research diagnoses for autism as determined by the lead clinicians (HHW and MLC) at each of the research sites. The best estimate diagnoses were made utilizing all available case material including clinical records, ADI-R results, and clinical assessment information. All qualified individuals met current DSM-IV diagnostic criteria for autism. The ADI-R (Lord et al. 1997) is a validated, semi-structured diagnostic interview, which yields a diagnostic algorithm based on the DSM-IV criteria for autism. All ADI-R interviews were conducted by formally trained interviewers who have established reliability. Finally, all participants who met current diagnostic criteria for autism were included only if they had a minimal developmental level of 18 months on the Vineland Adaptive Behavior Scale Score (Sparrow et al. 1984) or an IQ equivalent >35. These minimal developmental levels assure that ADI-R results are valid and reduce the likelihood of including individuals with severe mental retardation only. Subjects were excluded if they had evidence of developmental disorders with known phenotypic overlap with autism (e.g., Prader-Willi syndrome, Angelman syndrome, tuberous sclerosis complex, ReH Syndrome, and fragile X syndrome), neurologic, or severe sensory or motor disorders.

### Example 2

### Genotyping

Blood was obtained from patients and other family members according to IRB-approved procedures. DNA was extracted from whole blood using standard protocols (Vance 1998). Analysis of the candidate region was performed using data obtained from single nucleotide polymorphisms (SNPs). SNPs located within the GABAR subunit genes across chromosomes were analyzed. Between three and seven intronic and silent mutation SNPs within each gene were identified from Applied Biosystems ASSAYS ON DEMAND™ (AoD; ABI, Foster City, Calif.) products. The selected GABAR subunits (the number of SNPs typed) were GABRG1 (3), GABRA2 (6), GABRA4 (7) and GABRB1 (7) on 4p12; GABRB2 (6), GABRA6 (4), GABRA1 (5), GABRG2 (3) and GABRP (4) on 5q34-q35.1; GABRR1 (7) and GABRR2 (4) on 6q15; and GABRB3 (5), GABRA5 (4) and GABRG3 (5) on 15q12. SNPs were identified in the NCBI SNP database, and ordered as either ASSAYS-ON-DEMAND™ or ASSAYS-BY-DESIGN™ (Applied Biosystems, Foster City, Calif.). All SNPs were genotyped using TAQMAN®. All reactions contained 2.7 ng of total genomic DNA and were run on ABI 9700 GeneAmp PCR systems according to the manufacturer's instructions. Analysis of the SNP genotypes was performed using an ABI Prism® 7900HT Sequence Detection System (Applied Biosystems, Foster City, Calif.).

For quality control procedures, two CEPH standards were included on each 96-well plate, and samples from six individuals were duplicated across all plates as quality controls (QCs), with the laboratory technicians blinded to their identities. Analysis required that identical QC samples within and across plates had matching genotypes, in order to identify errors in loading and reading, and thus minimizing the error rate in genotype assignments. Meanwhile, a 95% efficiency of genotype is required. Technicians generating the genotypic data were blinded to the clinical statuses of the patients. After QC verification, genotypes of the samples were uploaded into the PEDIGENE® database and merged into the LAPIS management system for creating analysis input files (Haynes et al. 1995).

### Example 3

### Statistical Analysis

For further genotyping error checking, PedCheck (O'Connell J R et al., Amer. J. Hum. Genet. 63:259-266 (1998)) was run for Mendelian inheritance inconsistency detection. Merlin error checking (Abecasis et al. 2002) was run to identify the samples with excess recombinations and the families were checked further for possible genotyping error. A single affected and unaffected individual were selected randomly from each family for tests for Hardy-Weinberg Equilibrium (HWE), which was assessed using exact tests implemented in the Genetic Data Analysis program (Zaykin et al. 1995). For SNPs found to be out of HWE in the unaffected sample, a sequence of samples at that particular SNP was required to ensure the quality of the SNPs. Pair-wise linkage disequilibrium (D′ and r²) between markers was calculated using the GOLD® software package (Abecasis et al. 2000). The allelic association analyses were conducted using the pedigree disequilibrium test (PDT; Martin et al. 2000b) and the family based association test (FBAT; Horvath et al. 2004). These two tests are similar in many aspects, but each of them has distinct advantages. The PDT has the advantage of being valid as a test of both linkage and association in extended pedigrees, while the FBAT treats nuclear families within large pedigrees as independent, but permits haplotype-based association tests. Both PDT and FBAT are allele-based tests. The genotype-pedigree disequilibrium test (geno-PDT; Martin et al. 2003a) is an extension of PDT used to examine the association between marker genotype and disease. The haplotype family-based association test (HBAT; Horvath et al. 2004) was used for haplotype association analysis for SNPs within each GABAR subunit gene. Tagging SNPs within each gene were selected by using the confidence interval function in Haploview (Barrett et al. 2004). Both the haplotype-specific P-value and global P-value (adjustment for all possible haplotypes) were given in the program.

The core program of MDR (Ritchie et al. 2001; Hahn et al. 2003) was employed in this study to test for potential gene-gene interaction in order to identify specific locus combinations of interest for further investigation and replication. Some new features were added to the MDR through the extended MDR (EMDR) (Mei et al. 2005). Basically, the EMDR utilizes the same algorithm as the core MDR program, a data reduction program which tests for interactions (Ritchie et al. 2001; Hahn et al. 2003). The EMDR contains several new features. Briefly, these are 1) handling missing data in individuals with partial genotype data; 2) use of a Chi-square statistic in addition to the prediction error as a test statistic; and 3) introduction and implementation of a non-fixed permutation test to assess the statistical significance of models identified by EMDR. This non-fixed permutation generates an empirical P-value for a particular n-locus model considering all combinations of n loci. For example, for a particular 2-locus combination, the non-fixed permutation test accounts for the search of all possible 2-locus models to decide whether the best model is significant. An empirical P-value of less than 0.05 was regarded as statistically significant and is inherently adjusted for multiple testing. In this study, a cross-validation option was not utilized.

For case-control pairs used in EMDR, the proband (or most completely genotyped affected child) from each multiplex and triad family was selected (n=470 total) as a case and the untransmitted alleles were generated based on parental genotypes as a control. Given the sample size of 470 case-control pairs in this study, we did not test for interactions greater than 4-way (Mei et al. 2005).

Independent markers (tagging SNPs) were used in 4 by-chromosome models and selected by using the confidence interval function in Haploview (Barrett et al. 2004). Meanwhile, to retain adequate power to detect a gene-gene effect, the markers with the smallest P-values from 4 by-chromosome models were selected to build the final cross-chromosome model. The reason for the selection is that the permutation test inherently adjusts for multiple comparison and—true effects can be—overwhelmed when many markers are considered. Therefore, to maintain reasonable power a relatively small subset of markers was judiciously chosen for the MDR analysis. Each chromosome was examined and the markers having the smallest p-values within each chromosome were selected for the overall analysis.

The significant best models identified by EMDR can only suggest a gene-gene effect rather than a certain interaction. This holds especially true when a particular locus in a significant n-locus model also presents a significant main effect as the best 1-locus model. In this case, the identified gene-gene effect may be driven by the main effect from the locus rather than a true interaction. To verify the interaction between genes in the identified model, conditional logistic regression (using COXREG in SPSS version 11.5 for Windows [Cary, N.C., USA]) was performed. To test for interaction, all variables (markers in the identified model) and their interaction terms were forced into the model. The genotypes of the markers were recoded in logistic regression analysis. Genotypes with a case-to-control ratio of more than 1 were collapsed and recoded as the high-risk group, and those with the ratio less than 1 were recoded as the low-risk group. This matched the dimensionality reduction strategy applied in EMDR, enabling consistent interpretation of the results between the EMDR and logistic regression analysis. In this study, GG was coded as a high-risk group for marker RS1912960 and GG and TT were coded as high-risk groups for RS2351299. Finally, multi-locus geno-PDT and APL analysis (Martin et al. 2003b) were used to validate the gene-gene interaction from the logistic regression.

### Example 4

### Results

No significant deviation from HWE was found in unaffected Caucasians for all SNPs. SNP RS1426217 (GABRB3) on chromosome 15 presented evidence of deviation from HWE in the affected individuals (p=0.019). PDT showed that RS1912960 (GABRA4) on chromosome 4 had a preferential transmission of the common G allele to the affected offspring (p=0.012, Table I). In addition, FBAT identified a significant association at HCV9866022 (GABRR2) on chromosome 6 (p=0.04), where the PDT results suggested a similar trend (p=0.064) (the entire FBAT data are not shown; results were similar to PDT). Geno-PDT displayed positive genotype association with homozygous common genotypes TT, GG, and GG for HCV8262334 (GABRA2), RS1912960 (GABRA4), and RS2280073 (GABRA4), respectively, on chromosome 4 and with heterozygous genotypes CT and CG for RS2617503 and RS12187676 (GABRB2), respectively, on chromosome 5 (global-P shown in Table I). SNPs on the same chromosome did not show linkage disequilibrium with each other.

Haplotype analysis was performed using tagging SNPs within each gene and confirmed significant association with autism for specific haplotypes within GABRA2 (p=0.027), GABRA4 (p=0.025), and GABRR2 (p=0.028). However, the global P value (p>0.05) was not significant for any of the genes tested.

In order to test for a gene-gene effect, EMDR was run for chromosome-by-chromosome and cross-chromosome models. Out of all of the by-chromosome models (TABLES II-VI) that were tested, two significant models were found on chromosome 4. There is a 2-locus model involving RS1912960 in GABRA4 and RS2351299 in GABRB1 (p=0.002) and a 3-locus model involving RS2350439 in GABRG1, RS1912960 in GABRA4, and RS3114084 in GABRB1 (p=0.03), suggesting a potential gene-gene interaction among GABRG1, GABRA4, and GABRB1 (Table II). [Original MDR under 10-fold cross-validation option was run and confirmed a potential gene-gene effect in a 2-locus model (PE=43%, p=0.023).] From the cross-chromosome model (Table VI), EMDR identified the same best 1-locus and 2-locus (p=0.001) model as in the by-chromosome 4 model and confirmed the main effect at RS1912960 (GABRA4) (p=0.02). Another 3-locus model (RS282117 and RS2148174 in GABRR2 and RS208129 in GABRG3) (p=0.008) was also identified suggesting a potential gene-gene interaction across chromosomes between GABRR2 (Chr6) and GABRG3 (Chr15).

To evaluate whether the joint effects identified by the EMDR are the result of interacting genes, conditional logistic regression was conducted and the results supported a significant 2-locus gene-gene interaction between GABRA4 and GABRB1 (OR=2.9 for interaction term, high-risk vs. low risk, p=0.002), but did not detect an interaction in the cross-chromosome 3-locus or chromosome 4 3-locus model.

Consistent with the interaction term in the logistic regression described above (high-risk [GG] and high-risk [GG+TT] combination), multi-locus geno-PDT (Table VII) supported a positive cross-marker genotype association with disease between two common variant genotypes at RS1912960 [GG] and RS2351299 [GG]. The APL method confirmed a positive association from the G allele at RS1912960 (p=0.031) and also presented a positive haplotype association with disease from a haplotype with two common variants [G-G] (RS1912960 and RS2351299:p=0.014, Global p=0.014), which indirectly supported the genotype association shown in EMDR.

### Example 5

To the inventors' knowledge, this is the first comprehensive investigation of the allelic, genotypic, and haplotypic association together with the investigation of potential gene-gene interaction of all known autosomal GABAR subunit genes with autism. These novel findings indicate that GABRA4 is involved in the etiology of autism both independently and through interaction with GABRA1. These data support the hypothesis and present some of the first evidence that complex interactions account for autism risk.

In the present invention, several approaches were used to control for false positive results and thus to protect against incorrect conclusions regarding the etiology of the disease. First, only Caucasian autism families were included in the analysis in order to avoid biasing results due to population stratification. Second, all GABA genes selected have a substantial a priori probability of involvement in autism (Sullivan et al. 2001). Finally, a multi-analytic approach was used as previously described (Ashley-Koch et al. 2004) in order to interpret our findings. This approach looked for the convergence of results across several methods rather than relying on results from a single analytic tool. Specifically, several approaches were applied to validate the interaction identified by EMDR including conditional logistic regression.

To evaluate multi-locus effects in a comprehensive way, the results from allelic, genotypic, and haplotypic analyses were integrated for a best estimate. An extended version of MDR called EMDR (Mei et al. 2005) was also used in which several modifications were made to the MDR including allowing for missing data, improved estimation of test statistic distribution, and more accurate adjustment of multiple testing. These new features in EMDR have been previously validated (Mei et al. 2005). In this study, the no-cross validation option was chosen and the 10-fold cross-validation was omitted in each run. This option has shown a lower false positive and false negative rate than the original MDR (Mei et al. 2005).

Linkage and weak association to SNPs was previously reported in the cluster GABAR region on chromosome 15q in our autism data set (Menold et al. 2001; Martin et al. 2000a; Bass et al. 2000; Shao et al. 2003). One possible explanation for this finding may be that there are multiple disease variants for autism risk in this region and that any one variant is only weakly associated with an individual haplotype. Similarly, the present invention found no association with a single locus in this region. However, RS1426217, which is located in intron 6 of GABRB3, significantly deviated from HWE only in affected individuals. This does not invalidate the association analysis since both PDT and FBAT do not require HWE. Absence of HWE has previously been suggested to be an indication of the presence of association of a susceptibility allele that is in LD with the tested SNP (Nielsen et al. 1998). Thus, this finding might suggest that RS1426217 is in LD with a nearby disease allele. Extending the analysis to chromosome 15 GABAR genes (GABRB3, GABRA5, and GABRG3), a similar genetic analysis paradigm (Ashley-Koch et al. 2004) was applied to look for interactions amongst these three genes to determine if these interactions contribute to risk, but no multi-locus effects were detected. In a cross-chromosome model, however, a joint effect between GABRR2 (chromosome 6) and GABRG3 (chromosome 15) was found, although conditional logistic regression failed to confirm this interaction. Based on the FBAT and HBAT analysis results for GABRR2, this joint effect most likely is driven by the effect from GABRR2 only. Even so, the finding merits further investigation in a larger and/or independent sample.

The most promising finding in this study was the significant allelic and genotypic association that was found at RS1912960 (GABRA4) both from common variant G and common genotype GG. Also, HABT identified a significant haplotype within GABRA4 although the global P-value showed only marginal significance (p=0.06). Moreover, the association remained significant even after adjusting for multiple testing in EMDR. The program generates 1,000 simulated data sets by permuting the status of cases and controls to obtain an empirical P-value for the marker while testing the significance for the 1-locus best model. RS1912960 remained the best 1-locus model in both by-chromosome and cross-chromosome models. The empirical P-values for this marker were 0.038 and 0.020, respectively. Thus, this significant association appeared to be consistent across all analyses, strongly suggesting that GABRA4 is involved in the etiology of autism.

Relatively little is known about the biological function(s) of the α4 subunit. Gene expression is known to be highly variable depending upon brain region, neuronal activity, and development suggesting complex regulation and involvement in multiple brain activities and functions. Alpha4 mRNA levels are found in the hippocampus, dentate gyrus, thalamus, nucleus accumbens, cerebellum, the outer layers of the cortex, and other regions, and they peak during development. Unlike most GABAA receptor complexes, those containing a4 are not sensitive to modulation by diazepam. It has been suggested that the α4 subunit may be involved in neuronal hyperexcitability. The promoter for α4 has multiple transcription initiation sites, and alternate splicing in mouse brain has been observed (Ma et al. 2004).

Further, a potential interaction between GABRA4 and another clustering GABA gene, GABRB1 (OR=2.9 for interaction term) was found. This potential gene-gene effect model was identified in EMDR from both by-chromosome and cross-chromosome models. In addition, the interaction was further confirmed by conditional logistic regression, in which two common GG-GG variant combinations substantially increased autism risk. This finding is also consistent with the results from multi-locus geno-PDT (GG-GG) and APL haplotype analysis (G-G). Again, the culmination of findings across all analyses leads to the conclusion that GABRB1 may be involved through the interaction with GABRA4.

### Example 6

### GABA in Autism in Multiple Ethnic Groups

Despite similar prevalence rates between Caucasian and African Americans (Fombonne 2003b; Yeargin-Allsopp et al. 2003), autism studies in African Americans are rare. Risk alleles may be different between ethnic groups or the same risk alleles may have differential effects in each ethnic group, warranting studies in multiple groups. Evidence that phenotypic factors, including indicators of language development, may be more severe in African Americans, compared to non-Hispanic Caucasians, (Cuccaro et al. 2005) is consistent with these possibilities and underscores the need to investigate autism in different ethnic groups. Presented here is an independent dataset of 54 African American families, as well as an expanded Caucasian sample of 557 autism families.

Samples. All families were drawn from a large multi-site study of autism genetics conducted in the southeastern United States. These families are recruited through the Center for Human Genetics (CHG) at Duke. University Medical Center (DUMC), the University of South Carolina, and the Center for Human Genetic Research at Vanderbilt (N=54 African American and 557 Non-Hispanic Caucasian families) through support groups, advertisements, and clinical and educational settings. All sites recruited, enrolled, and sampled individuals with autism and family members, per study protocols approved by their respective institutional review boards (IRBs). Written informed consent was obtained from parents and from children who were able to give informed consent.

Families were enrolled based on probands meeting the following core inclusion criteria of: 1) probands ranging from three to 21 years of age; 2) a presumptive clinical diagnosis of autism; and 3) an expert clinical diagnosis of autism using DSM-IV criteria (American Psychiatric Association 1994), supported by the Autism Diagnostic Interview-Revised (ADI-R) (Rutter et al. 2003) and in some cases, the Autism Diagnostic Observation Schedule (ADOS) (Lord et al. 1999). To assure valid ADI-R results, all participants who met current diagnostic criteria for autism were included only if they had a minimal developmental level of 18 months, as extrapolated from the Vineland Adaptive Behavior Scale score (Sparrow et al. 1984), or had an IQ equivalent greater than 35. Exclusion criteria for participation in the larger genetics study included: severe sensory problems (e.g., visual impairment or hearing loss), significant motor impairments (e.g., failure to sit by 12 months, or walk by 24 months), or identified metabolic, genetic, or progressive neurological disorders, based on screening by clinical staff. Additional samples are from the Autism Genetic Research Exchange (AGRE).

Thirty-nine African American families were used in an initial GABA receptor screen. Follow-up analysis of significant findings was performed in 54 African American families. Analysis of the extended Caucasian dataset included 557 non-Hispanic Caucasian families. One-hundred and five new non-Hispanic Caucasian families were added to the analysis (18 families previously analyzed by Ma et al [20] were newly identified as Hispanic, and were omitted from the current study in an effort decrease heterogeneity in the Caucasian dataset).

Classification of history of seizure activity in autism patients was based on question 92 from the ADI-R, which queries for both current and lifetime presence of convulsions, seizures, and epilepsy. Caregiver responses to question 92 are coded to indicate no seizure activity, seizure activity with no definitive diagnosis of epilepsy, and seizures with a definite diagnosis of epilepsy. Using lifetime ratings, two groups of families were defined: those in which no seizure activity was reported, and those in which seizure activity was present in at least one autism patient. In addition, question 92 allows for coding of febrile seizures. Families with only febrile seizures were classified as negative for seizure activity and not included in the seizure subset analysis. Both families with positive and negative history of seizure activity were included in our overall dataset.

Molecular analyses and genotyping. The analysis of 14 GABA receptor subunit genes was performed in 39 African American families as previously described (Ma et al. 2005). Briefly, 70 SNPs within 14 GABA receptor genes on four autosomes were analyzed. Genes analyzed were: GABRA1, GABRA6, GABRB2, GABRG1, and GABRP from chromosome 5; GABRA2, GABRA4, GABRB1, and GABRG1 from chromosome 4; GABRB3, GABRA5, and GABRG3 from chromosome 15; and GABRR1 and GABRR2 from chromosome 6.

Additional SNPs within GABRA4 and GABRB1 were analyzed in the extended African American (N=54) and Caucasian (N=557) datasets to expand the coverage of variation across this region. Thirty-five SNPs, representative of different Linkage Disequilibrium (LD) blocks across the two genes (20 in GABRA4 and 15 in GABRB1), were genotyped. SNPs for genotyping were selected from online databases (University of California Santa Cruz and NCBI dbSNP) and from re-sequencing of exons and surrounding areas of both GABRB1 and GABRA4 genes.

SNP genotyping was performed using TAQMAN® allelic discrimination assays (Applied Biosystems). DNA was extracted from whole blood according to established protocols (Vance 1998), and 3 ng of genomic DNA was used per reaction. Amplification was performed on GeneAmp PCR Systems 9700 thermocyclers, with cycling conditions as recommended by Applied Biosystems. Fluorescence was measured using Applied Biosystem's 7900. Genotype discrimination was conducted using ABI Prism® SDS 2.1 software. Quality control, to ensure accurate genotyping, involved two different CEPH DNAs in quadruplicate on each 384 well plate, as well as the presence of samples which were replicated elsewhere in the sample list.

Statistical analysis. To ensure genotyping quality, Pedcheck was run for detection of Mendelian inheritance inconsistency. One affected and one unaffected individual from each family were selected randomly for tests of Hardy-Weinberg equilibrium (HWE), which was assessed using exact tests from the Genetic Data Analysis program (Zaykin et al. 1995). Pairwise Linkage Disequilibrium (LD) between markers was calculated using Graphical Overview of Linkage Disequilibrium (GOLDS®) (Abecasis et al. 2000) in the parents of autism cases for both the African American and Caucasian samples. LD was evaluated in parents in order to increase the available sample size for analysis and comparison between the two ethnic groups. The Pedigree Disequilibrium Test (PDT) and its extension the genotypic Pedigree Disequilibrium Test (genoPDT) (Martin et al. 2000b; Martin et al. 2003a) were used to test for association to autism susceptibility.

The EMDR (Ma et al. 2005; Mei et al. 2005), an extension of the MDR (Ritchie et al. 2001; Hahn et al. 2003), was used to test for potential gene-gene interaction, to identify specific locus combinations of interest for further investigation and validation of previous results. EMDR analysis was performed using seven SNPs, the four in GABRA4 found to show significant allelic or genotypic association in the Caucasian sample-set, and the three in GABRB1 found to be significant in the seizure subgroup. One, two, and three-way analysis was performed on the Caucasian dataset.

The Haplotype Family Based Association Test (HBAT; Horvath et al. 2004) was used for haplotype association analysis using the significant SNPs in GABRA4.

Results. Allelic association studies of 70 SNPs across the 14 GABA receptor subunit genes in the 39 African American screen set of families, revealed association in rs2280073 (GABRA4; p=0.0053) and hcv2119841 (GABRB1; p=0.0343), the same two genes identified through allelic association and interaction analysis in the Caucasian dataset [20]. Genotypic association analysis revealed the same GABRA4 SNP, rs2280073 (p=0.0262), and marginal significance within GABRP, rs1862242 (p=0.0471). The remaining SNPs showed no significant association (data not shown).

Analysis of the screening SNPs and newly identified SNPs within GABRA4 and GABRB1 in the Caucasian population (N=557), and within the extended African American population (N=54) (Table VII), revealed new SNPs with significant association. In the Caucasian dataset, rs1912960 increased in significance to p=0.0073. Additional significant SNPs were identified in GABRA4 as well, rs17599165 (p=0.0015) and rs17599416 (p=0.0040). Genotypic association was also seen in these SNPs (p=0.0046, 0.0009, and 0.0043 respectively), as well as in a fourth SNP, also in GABRA4 (rs7660336, p=0.0368). In the African American dataset, rs2280073 (p=0.0287), identified in the smaller African American dataset above, and rs16859788 (p=0.0253), were found to be associated with the allele based test. Genotypic association was also identified in rs16859788 (p=0.0412). No SNPs within GABRB1 were found to be associated with autism in either ethnic group.

The majority of pairwise r² values between the significant SNPs were less than 0.3, in both ethnic groups (Table IX). However, a few SNPs have values between 0.3 and 0.35. SNPs, rs17599165 and rs17599416, have r² values of 0.709 in African Americans and 0.853 in Caucasians, and rs7660336 and rs2280073 have a pairwise r² of 0.907 in Caucasians and 0.905 in African Americans. Allele frequencies are similar, yet not identical between the two groups. One SNP, however, showed almost no variation in the Caucasian dataset with a minor allele frequency of 0.24 in African Americans but only 0.001 in Caucasians (Table 2). Haplotype analysis, using the four SNPs with significant allelic or genotypic association in the Caucasian families, revealed a significant global test (p=0.014) in the Caucasian population, further supporting the involvement of these SNPs or another variant on the haplotype background.

Subsetting of the GABRA4 and GABRB1 data to analyze all families with positive history for seizures revealed no association to GABRA4. However, three SNPs within GABRB1 were found to be both allelically and genotypically associated with autism: rs2351299 (p=0.0163 and p=0.0189 for PDT and genoPDT respectively), rs4482737 (p=0.0339 and p=0.0339), and rs3832300 (p=0.0253 and p=0.0357). These three SNPs all had pairwise r2 values less than 0.1 (data not shown).

In the Caucasian population, EMDR verified the single locus effect identified through PDT analysis in rs1912960 (p=0.024), and identified two different significant two-locus gene-gene effects between GABRA4 and GABRB1, rs1912960 with rs2351299 (p=0.004), and rs17599416 with rs2351299 (p=0.014). Several three locus effects were also significant (rs7660336, rs1912960, and rs2351299 (p=0.012); rs17599165, rs1912960, and rs2351299 (p=0.012); rs1912960, rs17599416, and rs2351299 (p=0.038); and rs7660336, rs17599416, and rs2351299 (p=0.047)) (Table X). SNP rs2351299 is the same SNP identified in GABRB1 in the association studies of the seizure subset above.

The involvement of GABRA4 in autism has been confirmed through identification of significantly associated SNPs within an independent African American population. Furthermore, the original findings have been strengthened, including identification of additional associated SNPs and a significant interaction between GABRA4 and GABRB1 and in an extended dataset (N=557) of Caucasian autism families. The identification of association in GABRA4 in both the Caucasian and the African American datasets indicates that genetic variants within this gene are important to the genetic etiology of autism.

The identification of two different two-way interactions between GABRA4 and GABRB1 provides additional evidence of the complex interaction of these two genes in autism. The rs1912960 with rs2351299 interaction is between the same two SNPs described previously in the present application and reported in Ma et al. (2005) and is still significant in our larger dataset. However, the identification of an additional pair of SNPs, validates the initial finding of complex genetic interactions between these two genes. Given that one SNP, rs2351299, is in both pairs, it is possible that both pairs are being identified due to LD between the two GABRA4 SNPs (rs1912960 and rs17599416). Though the r2 value between these two SNPs (0.320) is not large, there is significant correlation. Given that these do not appear to be causative variants, it is likely that the true variant is yet to be identified, but is in LD with these GABRA4 SNPs. Examination of interaction in the independent dataset of African American families was not possible due to the limited sample size.

Variants within GABRB1 were also identified as associated within the autistic population with seizures. While no effect was seen in GABRA4, the sample size may be too small, given the potential but unknown effect, to conclude that it does or does not play a role in seizure status in autism. However, the enhanced findings in GABRB1 implicate seizure status as a potential subset in which GABRB1 contributes to genetic risk.

Despite the identification of GABRA4 in both ethnic groups, different SNPs were found to be associated. The identification of distinct SNPs within these populations may indicate differences in allele frequency and linkage disequilibrium within the two racial groups, differences in the haplotypic background in which identical causative variations originated, or differences in the causative variation. SNP rs16859788 for example, which is significant in the African American group, has practically no variation in the Caucasian dataset, therefore, providing no power for detection in this group. Other SNPs, however, show similar allele frequencies. Some differences in LD do exist between the two ethnic groups as well; however, the majority of the differences are small. The largest differences in LD are in pairwise values with rs16859788, which appear to mostly be due to the fact that the SNP is practically mono-allelic in the Caucasian population. The Caucasian dataset does suggest that there is a significant association of SNP haplotypes with risk, while the African American set does not. However, this difference may be due to the inability to pick up the haplotype association, due to the small size of the African American dataset. Therefore, while it is clear that minor allele frequency differences explain not identifying rs16859788 in the Caucasian dataset, additional studies are needed to try to identify all the reasons for the differences in the two ethnic groups.

While several associated SNPs have been identified, none of the ones in GABRA4 are predicted to have functional consequences; therefore, it is unlikely that these are primary variants leading to the autism susceptibility. One of the SNPs identified in GABRB1 in the seizure subset, however, is in the 3′ untranslated region (UTR). Given that multiple GABA receptor subunits combine in varying combinations to form a functional GABA receptor, even minor changes in levels of a particular subunit may alter the make up of receptors within a particular cell type, and alter the GABAergic signaling. Therefore, variations within potential regulatory regions, such as untranslated regions and promoters, could play an important role. It will be important to look at potential changes that may result from this and other potential GABRB1 UTR variations, as well as sequence coding and potential regulatory regions in order to identify the primary variation, or variations leading to altered autism susceptibility.

In summary, the GABA receptors are implicated in the etiology of autism in multiple ethnic populations, both independently and through complex interactions. These results validate our earlier findings, indicating GABRA4 and GABRB1 as genes contributing to autism susceptibility, extending these findings to multiple ethnic groups and suggesting seizures as a stratifying phenotype.

All publications, patent applications, patents, and other references mentioned herein are incorporated by reference in their entirety.

The foregoing is illustrative of the present invention, and is not to be construed as limiting thereof. The invention is defined by the following claims, with equivalents of the claims to be included therein.

TABLE I
The pedigree disequilibrium test (PDT) and Geno-PDT association
analysis of gamma aminobutyric acid (GABA) genes and autism

| | | | | Global-P | Global-P for |
|---|---|---|---|---|---|
| Chrom | No. | Gene | SNP | for PDT* | Geno-PDT^{#} |
| 4 | 1 | GABRG1 | RS1497571 | 0.899 | 0.906 |
| 4 | 2 | | RS2350439 | 0.509 | 0.717 |
| 4 | 3 | | RS1826923 | 0.340 | 0.622 |
| 4 | 4 | GABRA2 | HCV7537166 | 0.556 | 0.778 |
| 4 | 5 | | RS279858 | 0.361 | 0.623 |
| 4 | 6 | | RS279844 | 0.138 | 0.294 |
| 4 | 7 | | HCV8262290 | 0.064 | 0.141 |
| 4 | 8 | | RS4695152 | 0.508 | 0.730 |
| 4 | 9 | | HCV8262334 | 0.149 | 0.033 |
| 4 | 10 | GABRA4 | RS7678338 | 0.737 | 0.936 |
| 4 | 11 | | RS1512136 | 0.935 | 0.996 |
| 4 | 12 | | HCV1592545 | 1.000 | 0.968 |
| 4 | 13 | | RS1912960 | 0.012 | 0.003 |
| 4 | 14 | | RS2280073 | 0.072 | 0.034 |
| 4 | 15 | | RS10517174 | 0.738 | 0.142 |
| 4 | 16 | | RS3792211 | 0.677 | 0.391 |
| 4 | 17 | GABRB1 | RS2351299 | 0.817 | 0.098 |
| 4 | 18 | | RS1372496 | 0.088 | 0.160 |
| 4 | 19 | | RS3114084 | 0.180 | 0.317 |
| 4 | 20 | | HCV11353524 | 0.115 | 0.243 |
| 4 | 21 | | HCV2119841 | 0.906 | 0.432 |
| 4 | 22 | | RS6289 | 0.544 | 0.276 |
| 4 | 23 | | RS6290 | 0.940 | 0.506 |
| 5 | 24 | GABRB2 | RS253017 | 0.774 | 0.317 |
| 5 | 25 | | RS252965 | 0.649 | 0.299 |
| 5 | 26 | | RS2617503 | 0.108 | 0.025 |
| 5 | 27 | | RS2962425 | 0.367 | 0.443 |
| 5 | 28 | | RS2962407 | 0.771 | 0.149 |
| 5 | 29 | | RS12187676 | 0.407 | 0.015 |
| 5 | 30 | GABRA6 | RS3811995 | 0.613 | 0.488 |
| 5 | 31 | | RS6883829 | 0.932 | 0.236 |
| 5 | 32 | | HCV164095 | 0.814 | 0.920 |
| 5 | 33 | | RS3811991 | 0.652 | 0.283 |
| 5 | 34 | GABRA1 | RS4340950 | 0.426 | 0.650 |
| 5 | 35 | | HCV11258504 | 0.633 | 0.601 |
| 5 | 36 | | RS6878494 | 0.395 | 0.699 |
| 5 | 37 | | HCV1667770 | 0.861 | 0.522 |
| 5 | 38 | | HCV11814555 | 0.294 | 0.576 |
| 5 | 39 | GABRG2 | RS7728001 | 0.670 | 0.833 |
| 5 | 40 | | RS766349 | 0.700 | 0.223 |
| 5 | 41 | | RS211014 | 0.655 | 0.815 |
| 5 | 42 | GABRP | HCV3165046 | 0.872 | 0.779 |
| 5 | 43 | | RS1812910 | 0.965 | 0.981 |
| 5 | 44 | | RS1862242 | 0.347 | 0.593 |
| 5 | 45 | | RS1063310 | 0.560 | 0.807 |
| 6 | 46 | GABRR1 | RS404943 | 0.619 | 0.674 |
| 6 | 47 | | RS407206 | 0.623 | 0.835 |
| 6 | 48 | | RS423463 | 0.475 | 0.718 |
| 6 | 49 | | RS3777530 | 0.644 | 0.831 |
| 6 | 50 | | RS2297389 | 0.851 | 0.150 |
| 6 | 51 | | RS881293 | 0.832 | 0.978 |
| 6 | 52 | | RS6902106 | 0.829 | 0.712 |
| 6 | 53 | GABRR2 | RS282117 | 0.277 | 0.494 |
| 6 | 54 | | HCV9866022 | 0.064 | 0.171 |
| 6 | 55 | | RS2148174 | 0.855 | 0.770 |
| 6 | 56 | | HCV9865968 | 0.780 | 0.962 |
| 15 | 57 | GABRB3 | RS2081648 | 0.602 | 0.837 |
| 15 | 58 | | RS1426217 | 0.191 | 0.305 |
| 15 | 59 | | RS754185 | 0.672 | 0.852 |
| 15 | 60 | | HCV8865209 | 0.337 | 0.521 |
| 15 | 61 | | RS2059574 | 0.304 | 0.405 |
| 15 | 62 | GABRA5 | HCV42974 | 0.646 | 0.072 |
| 15 | 63 | | RS7173260 | 0.938 | 0.845 |
| 15 | 64 | | RS140681 | 0.886 | 0.762 |
| 15 | 65 | | RS140683 | 0.825 | 0.978 |
| 15 | 66 | GABRG3 | HCV2078506 | 0.079 | 0.240 |
| 15 | 67 | | RS208129 | 0.281 | 0.266 |
| 15 | 68 | | RS897173 | 0.240 | 0.451 |
| 15 | 69 | | HCV428306 | 1.000 | 0.611 |
| 15 | 70 | | RS140679 | 0.410 | 0.589 |

*P-value adjusted for 2 alleles
^{#}P-value adjusted for 3 genotypes

TABLE II
Best gene-gene effect models identified by extended multifactor dimensionality
reduction (EMDR) for gamma aminobutyric acid (GABA) receptor subunit genes on
chromosome 4

| | | | | | Chi- | |
|---|---|---|---|---|---|---|
| | | | | | squarenon- | Misclassification |
| Location | Genes | Marker | Marker-number | Best-model | fixed P^{a} | non-fixed P^{b} |
| 65.47 | GABRG1 | RS1497571 | 1 | 9 | 0.06 | 0.038 |
| 65.49 | | RS2350439 | 2 | 9 13 | 0.004 | 0.002^{c} |
| 65.66 | GABRA2 | RS279858 | 3 | 2 9 15 | 0.016 | 0.03 |
| 65.66 | | RS279844 | 4 | 2 7 9 15 | 0.16 | 0.33 |
| 65.67 | | HCV8262290 | 5 | | | |
| 65.68 | | RS4695152 | 6 | | | |
| 65.68 | GABRA4 | HCV8262334 | 7 | | | |
| 65.85 | | HCV1592545 | 8 | | | |
| 65.86 | | RS1912960 | 9 | | | |
| 65.86 | | RS2280073 | 10 | | | |
| 65.87 | | RS10517174 | 11 | | | |
| 65.87 | GABRB1 | RS3792211 | 12 | | | |
| 65.92 | | RS2351299 | 13 | | | |
| 65.94 | | RS1372496 | 14 | | | |
| 65.95 | | RS3114084 | 15 | | | |
| 65.97 | | HCV11353524 | 16 | | | |
| 65.99 | | HCV2119841 | 17 | | | |
| 66.00 | | RS6289 | 18 | | | |
| 66.00 | | RS6290 | 19 | | | |

^{a}empirical P-value derived from non-fixed permutation test by using chi-square as test statistic in EMDR
^{b}empirical P-value derived from non-fixed permutation test by using misclassification rate as test statistic in EMDR
^{c}locus (loci) with the lowest P-value (bold) are selected as the one into final cross-chromosome model

TABLE III
Best gene-gene effect models identified by extended multifactor dimensionality
reduction (EMDR) for gamma aminobutyric acid (GABA) receptor subunit genes on
chromosome 5

| | | | | | Chi-square | Misclassification |
|---|---|---|---|---|---|---|
| Location | Genes | Marker | Marker-number | Best-model | non-fixed P^{a} | non-fixed P^{b} |
| 165.09 | GABRB2 | RS253017 | 1 | 6 | 0.412 | 0.282^{c} |
| 165.09 | | RS252965 | 2 | 3 6 | 0.657 | 0.762 |
| 165.098 | | RS2617503 | 3 | 4 8 18 | 0.710 | 0.551 |
| 165.155 | | RS2962425 | 4 | 3 10 13 17 | 0.810 | 0.800 |
| 165.203 | | RS2962407 | 5 | | | |
| 165.246 | GABRA6 | RS12187676 | 6 | | | |
| 165.362 | | HCV164095 | 7 | | | |
| 165.364 | GABRA1 | RS3811991 | 8 | | | |
| 165.476 | | RS4340950 | 9 | | | |
| 165.494 | | RS6878494 | 10 | | | |
| 165.502 | | HCV1667770 | 11 | | | |
| 165.505 | GABRG2 | HCV11814555 | 12 | | | |
| 165.667 | | RS169793 | 13 | | | |
| 165.674 | | RS7728001 | 14 | | | |
| 165.677 | | RS766349 | 15 | | | |
| 165.693 | GABRP | RS211014 | 16 | | | |
| 182.846 | | HCV3165046 | 17 | | | |
| 182.859 | | RS1812910 | 18 | | | |
| 182.871 | | RS1862242 | 19 | | | |
| 182.877 | | RS1063310 | 20 | | | |

^{a}empirical P-value derived from non-fixed permutation test by using chi-square as test statistic in EMDR
^{b}empirical P-value derived from non-fixed permutation test by using misclassification rate as test statistic in EMDR
^{c}locus (loci) with the lowest P-value (bold) are selected as the one into final cross-chromosome model

TABLE IV
Best gene-gene effect models identified by extended multifactor dimensionality
reduction (EMDR) for gamma aminobutyric acid (GABA) receptor subunit genes on
chromosome 6

| | | | | | Chi-square | Misclassification |
|---|---|---|---|---|---|---|
| Location | Genes | Marker | Marker-number | Best-model | non-fixed P^{a} | non-fixed P^{b} |
| 94.926 | GABRR1 | RS404943 | 1 | 8 | 0.644 | 0.587 |
| 94.935 | | RS407206 | 2 | 5 11 | 0.579 | 0.452 |
| 94.946 | | RS423463 | 3 | 2 8 10 | 0.398 | 0.246^{c} |
| 94.975 | | RS3777530 | 4 | 7 8 10 11 | 0.645 | 0.665 |
| 94.991 | | RS2297389 | 5 | | | |
| 94.998 | | RS881293 | 6 | | | |
| 95.019 | | RS6902106 | 7 | | | |
| 95.171 | GABRR2 | RS282117 | 8 | | | |
| 95.208 | | HCV9866022 | 9 | | | |
| 95.238 | | RS2148174 | 10 | | | |
| 95.277 | | HCV9865968 | 11 | | | |

^{a}empirical P-value derived from non-fixed permutation test by using chi-square as test statistic in EMDR
^{b}empirical P-value derived from non-fixed permutation test by using misclassification rate as test statistic in EMDR
^{c}locus (loci) with the lowest P-value (bold) are selected as the one into final cross-chromosome model

TABLE V
Best gene-gene effect models identified by extended multifactor dimensionality
reduction (EMDR) for gamma aminobutyric acid (GABA) receptor subunit genes on
chromosome 15

| | | | | | Chi-square non- | Misclassification |
|---|---|---|---|---|---|---|
| Location | Genes | Marker | Marker-number | Best-model | fixed P^{a} | non-fixed P^{b} |
| 11.07 | GABRB3 | RS2081648 | 1 | 10 | 0.219^{c} | 0.706 |
| 11.08 | | RS1426217 | 2 | 5 10 | 0.494 | 0.56 |
| 11.23 | | RS754185 | 3 | 4 10 13 | 0.843 | 0.85 |
| 11.33 | | HCV8865209 | 4 | 4 5 10 13 | 0.623 | 0.875 |
| 11.54 | | RS2059574 | 5 | | | |
| 12.06 | GABRA5 | HCV42974 | 6 | | | |
| 12.12 | | RS140681 | 7 | | | |
| 12.14 | | RS140683 | 8 | | | |
| 12.38 | GABRG3 | HCV2078506 | 9 | | | |
| 12.81 | | RS208129 | 10 | | | |
| 12.94 | | RS897173 | 11 | | | |
| 14.46 | | HCV428306 | 12 | | | |
| 14.66 | | RS140679 | 13 | | | |

^{a}empirical P-value derived from non-fixed permutation test by using chi-square as test statistic in EMDR
^{b}empirical P-value derived from non-fixed permutation test by using misclassification rate as test statistic in EMDR
^{c}locus (loci) with the lowest P-value (bold) are selected as the one into final cross-chromosome model

TABLE VI
Best gene-gene effect models identified by extended multifactor dimensionality
reduction (EMDR) for all known autosomal gamma aminobutyric acid (GABA) receptor
subunit genes

| | | | Chrom-Marker | | | Chi-square | Misclassification |
|---|---|---|---|---|---|---|---|
| Location | Genes | Marker | number | SNP No. | Best-model | non-fixed P^{a} | non-fixed P^{b} |
| 65.857 | GABRA4 | RS1912960 | 4-9 | 1 | 1 | 0.035 | 0.02 |
| 65.916 | GABRB1 | RS2351299 | 4-13 | 2 | 1 2 | 0.002 | 0.001 |
| 165.246 | GABRB2 | RS12187676 | 5-6 | 3 | 5 6 7 | 0.009 | 0.008 |
| 94.935 | GABRR1 | RS407206 | 6-2 | 4 | | | |
| 95.171 | GABRR2 | RS282117 | 6-8 | 5 | | | |
| 95.238 | GABRR2 | RS2148174 | 6-10 | 6 | | | |
| 12.813 | GABRG3 | RS208129 | 15-10 | 7 | | | |

^{a}empirical P-value derived from non-fixed permutation test by using chi-square as test statistic in EMDR
^{b}empirical P-value derived from non-fixed permutation test by using misclassification rate as test statistic in EMDR

TABLE VII
Results for multi-locus genotype pedigree disequilibrium
test (geno-PDT) between 2-loci in chromosome 4

| Genotype-RS1912960^{a} | Genotype-RS2351299^{a} | P-value^{b} |
|---|---|---|
| 1, 1 | 1, 1 | 0.015 |
| 1, 1 | 1, 2 | 0.330 |
| 1, 1 | 2, 2 | 0.096 |
| 1, 2 | 1, 1 | 0.061 |
| 1, 2 | 1, 2 | 0.635 |
| 1, 2 | 2, 2 | 0.835 |
| 2, 2 | 1, 1 | 0.001 |
| 2, 2 | 1, 2 | 0.046 |
| 2, 2 | 2, 2 | 0.386 |
| | Global P-value^{c} | 0.0007 |

^{a}RS1912960: 1: C; 2: G (common allele); RS2351299: 1: G (common allele); 2: T
^{b}P-value for each genotype combination;
^{c}Global P-value: after adjusted for all possible genotype combinations

TABLE VIII
Analysis of GABRA4 and GABRB1 in extended Caucasian and African
American datasets

| | GABRA4 | | | | GABRB1 | | | | |
|---|---|---|---|---|---|---|---|---|---|
| | | | African | | | | | African | |
| | Caucasian | | American | | | Caucasian | | American | |
| | | Geno | | Geno | | | Geno | | Geno |
| SNP | PDT^{a} | PDT^{a} | PDT^{a} | PDT^{a} | SNP | PDT | PDT | PDT | PDT |
| RS7678338 | 0.9350 | 0.9923 | 0.2230 | 0.4190 | RS1866989 | 0.3071 | 0.3860 | 0.5553 | 0.7892 |
| RS6447517 | 0.8826 | 0.7034 | 0.505 | 0.3930 | RS2351299 | 0.4529 | 0.0822 | 0.5775 | 0.8614 |
| RS17599102 | 0.8055 | 0.8913 | 0.7518 | 0.8805 | RS10016388 | 0.1585 | 0.2660 | 0.2367 | 0.4259 |
| RS7660336 | 0.0833 | 0.0368(G/G) | 0.5164 | 0.7410 | RS1372496 | 0.2362 | 0.3740 | 0.2482 | 0.3715 |
| RS1512136 | 0.9052 | 0.9869 | 0.2888 | 0.3575 | RS3114084 | 0.0934 | 0.1942 | 0.2059 | 0.3281 |
| RS17599165 | 0.0015(T) | 0.0009(T/T) | 0.6547 | 0.4304 | RS4482737 | 0.1495 | 0.2504 | 1.0000 | 1.0000 |
| HCV1592545 | 0.7798 | 0.9427 | 0.2230 | 0.5313 | HCV11353524 | 0.1959 | 0.3117 | 1.0000 | 1.0000 |
| RS7685553 | 1.0000 | 0.8419 | 0.4913 | 0.6044 | RS3775534 | 0.1893 | 0.1831 | 0.4913 | 0.4913 |
| RS1912960 | 0.0073(C) | 0.0046(C/C) | 0.4111 | 0.5110 | HCV2119841 | 0.3352 | 0.0838 | 0.2278 | 0.4111 |
| RS2055943 | 0.9671 | 0.9434 | 0.4927 | 0.7607 | RS6287 | 0.4045 | 0.3571 | 0.6171 | 0.6984 |
| RS2280073 | 0.1404 | 0.0955 | 0.0287(G) | 0.1100 | RS6289 | 0.9349 | 0.5554 | 0.8658 | 0.9220 |
| RS16859788 | 0.3173 | 0.3173 | 0.0253(A) | 0.0412(A/A) | RS6290 | 0.4285 | 0.1973 | 0.3173 | 0.4594 |
| RS17599416 | 0.0040(A) | 0.0043(A/A) | 0.8084 | 0.8084 | 4P0413 | 1.0000 | 1.0000 | 0.7389 | 0.7389 |
| RS3792208 | 1.0000 | 0.4980 | 0.1797 | 0.1797 | RS10028945 | 0.3272 | 0.4584 | 1.0000 | 0.8179 |
| RS10517174 | 0.9484 | 0.0894 | 0.7150 | 0.8903 | RS3832300 | 0.4094 | 0.6352 | 0.6547 | 0.6547 |
| RS7694035 | 0.4337 | 0.6266 | 0.3657 | 0.3657 | | | | | |
| RS3792211 | 0.9057 | 0.8382 | 0.6547 | 0.2895 | | | | | |
| RS2229940 | 0.7873 | 0.9375 | 0.5485 | 0.7866 | | | | | |
| RS13151759 | 0.7529 | 0.9326 | 0.2367 | 0.5759 | | | | | |
| RS13151769 | 0.4740 | 0.7436 | 0.1824 | 0.4768 | | | | | |

^{a}Associated allele/genotype shown in parenthesis

TABLE IX
Minor allele frequencies and Linkage Disequilibrium in Caucasian and African
American datasets

| | MAF^{a} | | | | | | | |
|---|---|---|---|---|---|---|---|---|
| | | African | | | | | | |
| SNP | Caucasian | American | | RS7660336 | RS17599165 | RS1912960 | RS2280073 | RS16859788 |
| RS7660336 RS17599165 RS1912960 RS2280073 RS16859788 RS17599416 RS2351299 RS4482737 RS3832300 | 0.500 0.085 0.228 0.500 0.0010.100 0.178 0.012 0.044 | 0.418 0.073 0.217 0.410 0.2400.06 0.275 0.041 0.061 | African American R² | 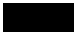 0.098 0.376 0.905 0.236 0.096 0.09 0.003 0.019 | | | | |
| # 0.102 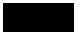 0.312 0.06 0.022 0.709 0.011 0.004 0.002 | 0.286 0.331 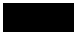 0.342 0.085 0.301 0.006 0 0 | 0.907 0.097 0.301 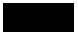 0.223 0.104 0.114 0.008 0.019 | .001 0 0 0 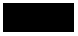 0.022 0.064 0.013 0 | | | | | |

| | MAF^{a} | | | | | |
|---|---|---|---|---|---|---|
| | | African | | | | |
| SNP | Caucasian | American | RS17599416 | RS2351299 | RS4482737 | RS3832300 |
| RS7660336 RS17599165 RS1912960 RS2280073 RS16859788 RS17599416 RS2351299 RS4482737 RS3832300 | 0.500 0.085 0.228 0.500 0.0010.100 0.178 0.012 0.044 | 0.418 0.073 0.217 0.410 0.2400.06 0.275 0.041 0.061 | 0.104 0.853 0.320 0.113 0 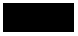 0.007 0.002 0.005 | | | |
| # 0 0.008 0.003 0 0 0.012 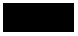 0.057 0.018 | 0.001 0 0 0.001 0 0 0.002 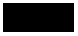 0.019 | 0.009 0 0 0.008 0 0 0 0.04 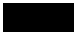 | Caucasian r² | | | |

^{a}MAF = minor allele frequency

TABLE X
EMDR results in Caucasian dataset between GABRA4
and GABRB1

| Input SNPs | | | Significant Interactions | | |
|---|---|---|---|---|---|
| Gene | SNP number | SNP | | SNPs | P-values |
| GABRA4 | 1 | RS7660336 | One-way | 3 | 0.024 |
| | 2 | RS17599165 | Two-way | 3, 5 | 0.004 |
| | 3 | RS1912960 | | 4, 5 | 0.014 |
| | 4 | RS17599416 | Three-way | 1, 3, 5 | 0.012 |
| GABRB1 | 5 | RS2351299 | | 2, 3, 5 | 0.012 |
| | 6 | RS4482737 | | 3, 4, 5 | 0.038 |
| | 7 | RS3832300 | | 1, 4, 5 | 0.047 |

### REFERENCES

Abecasis G R, Cherny S S, Cookson W O, and Cardon L R (2002) Merlin—rapid analysis of dense genetic maps using sparse gene flow trees. Nat Genet 30:97-101
Abecasis G R and Cookson W O (2000) GOLD—graphical overview of linkage disequilibrium. BioInformatics 16:182-183
Aldred S, Moore K M, Fitzgerald M, and Waring R H (2003) Plasma amino acid levels in children with autism and their families. J Autism Dev Disord 33:93-97
American Psychiatric Association. 1994. Diagnostic and Statistical Manual of Mental Disorders (DSM-IV). Vol. IV. Frances ATFC (ed). Washington, D.C.: American Psychiatric Press, Inc.
Ashley-Koch A E, Martin E R, Jaworski J, Ma D Q, Rabionet R, Skaar D A, Menold M M, Abramson R K, Wright H H, Cuccaro M L, Gilbert J R, and Pericak-Vance M A (2004) Evidence for gene-gene interactions influencing susceptibility to autistic disorder. XIIth World Congress of Psychiatric Genetics Dublin, Ireland, 9th-13th October
Auranen M, Vanhala R, Varilo T, Ayers K, Kempas E, Ylisaukko-Oja T, Sinsheimer J S, Peltonen L, and Jarvela I, 1 (2002) A Genomewide Screen for Autism-Spectrum Disorders: Evidence for a Major Susceptibility Locus on Chromosome 3q25-27. Am J Hum Genet 71:777-790
Bailey A, Le Couteur A, Gottesman I, Bolton P, Simonoff E, Yuzda E, and Rutter M (1995) Autism as a strongly genetic disorder: evidence from a British twin study. Psychol Med 25:63-77
Barrett J C, Fry B, Maller J, and Daly M J (2004) Haploview: analysis and visualization of LD and haplotype maps. Bioinformatics
Bass M P, Menold M M, Wolpert C M Donnelly S L, Ravan S A, Hauser E R, Maddox L O, Vance J M, Abramson R K, Wright H H, Gilbert J R, Cuccaro M L, DeLong G R, and Pericak-Vance M A (2000) Genetic studies in autistic disorder and chromosome 15. Neurogenetics 2:219-226
Blatt G J, Fitzgerald C M, Guptill J T, Booker A B, Kemper T L, and Bauman M L (2001) Density and distribution of hippocampal neurotransmitter receptors in autism: an autoradiographic study. J Autism Dev Disord 31:537-543
Bolton P, Macdonald H, Pickles A, Rios P, Goode S, Crowson M, Bailey A, and Rutter M (1994) A case-control family history study of autism. Journal of Child Psychology & Psychiatry & Allied Disciplines 35:877-900
Boyar F Z, Whitney M M, Lossie A C, Gray B A, Keller K L, Stalker H J, Zori R T, Geffken G, Mutch J, Edge P J, Voeller K S, Williams C A, and Driscoll D J (2001) A family with a grand-maternally derived interstitial duplication of proximal 15q. Clin Genet 60:421-430
Bundey S, Hardy C, Vickers S, Kilpatrick M W, and Corbett J A (1994) Duplication of the 15q11-13 region in a patient with autism, epilepsy and ataxia. Dev Med Child Neurol 36:736-742
Buxbaum J D, Silverman J M, Smith C J, Greenberg D A, Kilifarski M, Reichert J, Cook E H, Jr., Fang Y, Song C Y, and Vitale R (2002) Association between a GABRB3 polymorphism and autism. Mol Psychiatry 7:311-316
Chugani D C, Muzik O, Juhasz C, Janisse J J, Ager J, and Chugani H T (2001) Postnatal maturation of human GABAA receptors measured with positron emission tomography. Ann Neurol 49:618-626
Coffey C S, Hebert P R, Ritchie M D, Krumholz H M, Gaziano J M, Ridker P M, Brown N J, Vaughan D E, and Moore J H (2004) An application of conditional logistic regression and multifactor dimensionality reduction for detecting gene-gene interactions on risk of myocardial infarction: the importance of model validation. BMC Bioinformatics 5:49
Collaborative Linkage Study of Autism (2001) An autosomal genomic screen for autism. Am J Med Genet 105:609-615
Cook E H, Jr., Courchesne R Y, Cox N J, Lord C, Gonen D, Guter S J, Lincoln A, Nix K, Haas R, Leventhal B L, and Courchesne E (1998) Linkage-disequilibrium mapping of autistic disorder, with 15q11-13 markers. Am J Hum Genet 62:1077-1083
Cuccaro M L, Donnelly S, Cope H, Wolpert C, Carney R, Abramson R K, Hall A, Wright H H, Gilbert J R, Pericak-Vance M A (2005) Autism in African American (AA) families: Phenotypic findings. American Society of Human Genetics 55th Annual Meeting, Salt Lake City, Utah
Dhossche D, Applegate H, Abraham A, Maertens P, Bland L, Bencsath A, and Martinez J (2002) Elevated plasma gamma-aminobutyric acid (GABA) levels in autistic youngsters: stimulus for a GABA hypothesis of autism. Med Sci Monit 8:R1-R6
Folstein S E, and Rosen-Sheidley B (2001) Genetics of autism: complex aetiology for a heterogeneous disorder. Nat Rev Genet 2:943-955
Fombonne E (1999) The epidemiology of autism: a review. Psychol Med 29:769-786
Fombonne E (2003a) Epidemiological surveys of autism and other pervasive developmental disorders: an update. J Autism Dev Disord 33:365-382
Fombonne E (2003b) The prevalence of autism. JAMA 289:87-89.
Hahn L W, Ritchie M D, and Moore J H (2003) Multifactor dimensionality reduction software for detecting gene-gene and gene-environment interactions. Bioinformatics 19:376-382
Haynes C, Speer M C, Peedin M, Roses A D, Haines J L, Vance J M, and Pericak-Vance M A (1995) PEDIGENE: A comprehensive data management system to facilitate efficient and rapid disease gene mapping. Am J Hum Genet 57:A193
Horvath S, Xu X, Lake S L, Silverman E K, Weiss S T, and Laird N M (2004) Family-based tests for associating haplotypes with general phenotype data: application to asthma genetics. Genet Epidemiol 26:61-69
Hussman J P (2001) Suppressed GABAergic inhibition as a common factor in suspected etiologies of autism. Journal of Autism and Developmental Disorders 31:247-248
International Molecular Genetic Study of Autism Consortium (1998) A full genome screen for autism with evidence for linkage to a region on chromosome 7q. Hum Mol Genet 7:571-578
International Molecular Genetic Study of Autism Consortium (2001) A genomewide screen for autism: strong evidence for linkage to chromosomes 2q, 7q, and 16p. Am J Hum Genet 69:570-581
Jentsch T J, Stein V, Weinreich F, and Zdebik A A (2002) Molecular structure and physiological function of chloride channels. Physiol Rev 82:503-568
Liu J, Nyholt D R, Magnussen P, Parano E, Pavone P, Geschwind D, Lord C, Iversen P, Hoh J, Ott J, and Gilliam T C (2001) A genomewide screen for autism susceptibility loci. Am J Hum Genet 69:327-340
Lord C, Pickles A, McLennan J, Rutter M, Bregman J, Folstein S, Fombonne E, Leboyer M, and Minshew N (1997) Diagnosing autism: analyses of data from the Autism Diagnostic Interview. J Autism Dev Disord 27:501-517
Lord C, Rutter M, DiLavore P, Risi S. Autism Diagnostic Observation Schedule-WPS (WPS Edition). 1999. Los Angeles, Western Psychological Association.
Ma L, Song L, Radoi G E, and Harrison N L (2004) Transcriptional regulation of the mouse gene encoding the alpha-4 subunit of the GABAA receptor. J Biol Chem 279:40451-40461
Ma D Q, Whitehead P L, Menold M M, Martin E R, Ashley-Koch A E, Mei H, Ritchie M D, DeLong G R, Abramson R K, Wright H H, Cuccaro M L, Hussman J P, Gilbert J R, Pericak-Vance M A (2005) Identification of Significant Association and Gene-Gene Interaction of GABA Receptor Subunit Genes in Autism. Am J Hum Genet 77:377-388.
Macdonald R L, Gallagher M J, Feng H J, Kang J (2004) GABA(A) receptor epilepsy mutations. Biochem Pharmacol 68:1497-1506.
Martin E R, Bass M P, Gilbert J R, Pericak-Vance M A, and Hauser E R (2003a) Genotype-based association test for general pedigrees: the genotype-PDT. Genet Epidemiol 25:203-213
Martin E R, Bass M P, Hauser E R, and Kaplan N L (2003b) Accounting for linkage in family-based tests of association with missing parental genotypes. Am J Hum Genet 73:1016-1026 Martin E R, Menold M M, Wolpert C M, Bass M P, Donnelly S L, Ravan S A, Zimmerman A, Gilbert J R, Vance J M, Maddox L O, Wright H H, Abramson R K, DeLong G R, Cuccaro M L, and Pericak-Vance M A (2000a) Analysis of linkage disequilibrium in gamma-aminobutyric acid receptor subunit genes in autistic disorder. Am J Med Genet 96:43-48
Martin E R, Monks S A, Warren L L, and Kaplan N L (2000b) A test for linkage and association in general pedigrees: the pedigree disequilibrium test. Am J Hum Genet 67:146-154
Mei H, Ma D Q, Ashley-Koch A E, Martin E R. Extension of Multifactor Dimensionality Reduction for identifying multi-locus effects in the GAW14 simulated data. BMC Genetics 6(Suppl 1).
Menold M M, Shao Y, Wolpert C M, Donnelly S L, Raiford K L, Martin E R, Ravan S A, Abramson R K, Wright H H, DeLong G R, Cuccaro M L, Pericak-Vance M A, and Gilbert J R (2001) Association analysis of chromosome 15 gaba receptor subunit genes in autistic disorder. J Neurogenet 1.5:245-259
Meyers R M, Risch N, Spiker D, and Lotspeich L (1998) A Full Genome Screen for Susceptibility Genes to Autism by Linkage Analysis in Ninety Multiplex Families. Am J Hum Genet 63:A302
Moore J H (2003) The ubiquitous nature of epistasis in determining susceptibility to common human diseases. Hum Hered 56:73-82
Moore J H (2005) A global view of epistasis. Nat Genet 37:13-14
Moore J H, and Williams S M (2002) New strategies for identifying gene-gene interactions in hypertension. Ann Med 34:88-95
Muhle R, Trentacoste S V, and Rapin 1 (2004) The genetics of autism. Pediatrics 113:e472-e486
Nielsen D M, Ehm M G, and Weir B S (1998) Detecting marker-disease association by testing for Hardy-Weinberg disequilibrium at a marker locus. Am J Hum Genet 63:1531-1540
O'Connell J R et al., Amer. J. Hum. Genet. 63:259-266 (1998))
Philippe A, Martinez M, Guilloud-Bataille M, Gillberg C, Rastam M, Sponheim E, Coleman M, Zappella M, Aschauer H, Van Maldergem L, Penet C, Feingold J, Brice A, Leboyer M, and van Malldergerme L (1999) Genome-wide scan for autism susceptibility genes. Paris Autism Research International Sibpair Study. Hum Mol Genet 8:805-812
Pickles A, Bolton P, Macdonald H, Bailey A, Le Couteur A, Sim C H, and Rutter M (1995) Latent-class analysis of recurrence risks for complex phenotypes with selection and measurement error: a twin and family history study of autism. Am J Hum Genet 57:717-726
Pritchard J K (2001) Are rare variants responsible for susceptibility to complex diseases? Am J Hum Genet 69:124-137
Pujana M A, Nadal M, Guitart M, Armengol L, Gratacos M, and Estivill X (2002) Human chromosome 15q11-q14 regions of rearrangements contain clusters of LCR15 duplicons. Eur J Hum Genet 10:26-35
Risch N, Spiker D, Lotspeich L, Nouri N, Hinds D, Hallmayer J, Kalaydjieva L et al (1999) A genomic screen of autism: evidence for a multilocus etiology. Am J Hum Genet 65:493-507
Ritchie M D, Hahn L W, Roodi N, Bailey L R, Dupont W D, Parl F F, and Moore J H (2001) Multifactor-dimensionality reduction reveals high-order interactions among estrogen-metabolism genes in sporadic breast cancer. Am J Hum Genet 69:138-147
Rolf L H, Haarmann F Y, Grotemeyer K H, Kehrer H (1993) Serotonin and amino acid content in platelets of autistic children. Acta Psychiatr Scand 87:312-316.
Rutter M, LeCouteur A, Lord C. Autism Diagnostic Interview, Revised (ADI-R). 2003. Los Angeles, Calif., Western Psychological Services.
Shao Y, Cuccaro M L, Hauser E R, Raiford K L, Menold M M, Wolpert C M, Ravan S A, Elston L, Decena K, Donnelly S L, Abramson R K, Wright H H, DeLong G R, Gilbert J R, and Pericak-Vance M A (2003) Fine mapping of autistic disorder to chromosome 15q11-q13 by use of phenotypic subtypes. Am J Hum Genet 72:539-548
Shao Y, Wolpert C M, Raiford K L, Menold M M, Donnelly S L, Ravan S A, Bass M P, McClain C, von Wendt L, Vance J M, Abramson R H, Wright H H, Ashley-Koch A, Gilbert J R, DeLong R G, Cuccaro M L, and Pericak-Vance M A (2002a) Genomic screen and follow-up analysis for autistic disorder. Am J Med Genet 114:99-105
Shao Y, Cuccaro M L, Hauser E R, Raiford K L, Wolpert C M, Donnelly S L, Elston L, Ravan S A, Abramson R K, Wright H H, DeLong G R, Gilbert J R, Pericak-Vance M A (2002b) Linkage of Autistic Disorder to chromosome 15q11-q13 using phenotypic subtypes. American Society of Human Genetics Annual Meeting:A1669.
Smith M, Filipek P A, Wu C, Bocian M, Hakim S, Modahl C, and Spence M A (2000) Analysis of a 1-megabase deletion in 15q22-q23 in an autistic patient: identification of candidate genes for autism and of homologous DNA segments in 15q22-q23 and 15q11-q13. Am J Med Genet 96:765-770
Sparrow S S, Balla D, Cicchetti D. 1984. Vineland Adaptive Behavior Scales, Interview Edition. Circle Pines, Minn.: AGS Publishing
Steffenburg S, Gillberg C, Hellgren L, Andersson L, Gillberg I C, Jakobsson G, and Bohman M (1989) A twin study of autism in Denmark, Finland, Iceland, Norway, and Sweden. J Child Psychol Psychiatry 30:405-416
Sullivan P F, Eaves L J, Kendler K S, and Neale M C (2001) Genetic case-control association studies in neuropsychiatry. Arch Gen Psychiatry 58:1015-1024
Thornton-Wells T A, Moore J H, and Haines J L (2004) Genetics, statistics and human disease: analytical retooling for complexity. Trends Genet 20:640-647
Vance J M (1998) The Collection of Biological Samples for DNA Analysis. In Haines J L, Pericak-Vance M A (eds) Approaches to Gene Mapping in Complex Human Diseases. New York: Wiley-Liss
Veenstra-VanderWeele J, Christian S L, and Cook E H, Jr. (2004) Autism as a paradigmatic complex genetic disorder. Annu Rev Genomics Hum Genet 5:379-405
Wolpert C M, Menold M M, Bass M P, Qumsiyeh M B, Donnelly S L, Ravan S A, Vance J M, Gilbert J R, Abramson R K, Wright H H, Cuccaro M L, and Pericak-Vance M A (2000) Three probands with autistic disorder and isodicentric chromosome 15. Am J Med Genet 96:365-372
Yeargin-Allsopp M, Rice C, Karapurkar T, Doernberg N, Boyle C, and Murphy C (2003) Prevalence of autism in a US metropolitan area. JAMA 289:49-55
Yonan A L, Alarcon M, Cheng R, Magnusson P K E, Spence S J, Palmer A A, Grunn A, Juo S H H, Terwilliger J D, Liu J J, Cantor R M, Geschwind D H, and Gilliam T C (2003) A genomewide screen of 345 families for autism-susceptibility loci. Am J Hum Genet 73:886-897
Zaykin D, Zhivotovsky L, and Weir B S (1995) Exact tests for association between alleles at arbitrary numbers of loci. Genetica 96:169-178

That which is claimed:

1. A method of identifying a subject having an increased risk of developing an autistic disorder, comprising detecting in the subject one or more genetic markers within a gamma-aminobutyric acid receptor (GABAR) subunit gene correlated with an increased risk of developing an autistic disorder.

2. A method of identifying a subject having an increased risk of developing an autistic disorder, comprising:
a) correlating the presence of one or more genetic markers within a GABAR subunit gene with an increased risk of developing autistic disorder; and
b) detecting the one or more genetic markers of step (a) in the subject, thereby identifying the subject as having an increased risk of developing autistic disorder.

3. The method of claim 1, wherein the genetic marker is selected from the group consisting of a single nucleotide polymorphism within a gamma-aminobutyric acid receptor, alpha-4 (GABRA4) gene, a single nucleotide polymorphism within a gamma-aminobutyric acid receptor, alpha-2 (GABRA2) gene, a single nucleotide polymorphism within a gamma-aminobutyric acid receptor, beta-1 (GABRB1) gene, a single nucleotide polymorphism within a gamma-aminobutyric acid receptor, beta-2 (GABRB2) gene, a single nucleotide polymorphism within a gamma-aminobutyric acid receptor, beta-3 (GABRB3) gene, a single nucleotide polymorphism within a gamma-aminobutyric acid receptor, pi (GABRP) gene, a single nucleotide polymorphism within a gamma-aminobutyric acid receptor, rho-2 (GABRR2) gene, a single nucleotide polymorphism within a gamma-aminobutyric acid receptor, gamma 1 (GABRG1) gene, a single nucleotide polymorphism within a gamma-aminobutyric acid receptor, gamma 3 (GABRG3) gene and any combination thereof.

4. The method of claim 3, wherein the single nucleotide polymorphism within the GABRA4 gene is selected from the group consisting of rs1912960, rs2280073, rs17599165, rs17599416, rs7660336, rs16859788, and any combination thereof.

5. The method of claim 3, wherein the single nucleotide polymorphism within the GABRB1 gene is selected from the group consisting of hcv2119841, rs2351299, rs4482737, rs383230, RS3114084, and any combination thereof.

6. The method of claim 3, wherein the single nucleotide polymorphism within the GABRB2 gene is selected from the group consisting of RS2617503, RS12187676, and a combination thereof.

7. The method of claim 3, wherein the single nucleotide polymorphism within the GABRB3 gene is RS1426217.

8. The method of claim 3, wherein the single nucleotide polymorphism within the GABRP gene is rs1862242.

9. The method of claim 3, wherein the single nucleotide polymorphism within the GABRA2 gene is HCV8262334.

10. The method of claim 3, wherein the single nucleotide polymorphism within the GABRR2 gene is HCV9866022, RS2148174 and RS2822117.

11. The method of claim 3, wherein the single nucleotide polymorphism within the GABRG1 gene is RS2350439.

12. The method of claim 3, wherein the single nucleotide polymorphism within the GABRG3 gene is RS208129.

13. The method of claim 3, wherein the genetic marker is a combination of the single nucleotide polymorphism rs1912960 within the GABRA4 gene and the single nucleotide polymorphism rs2351299 within the GABRB1 gene.

14. The method of claim 3, wherein the genetic marker is a combination of the single nucleotide polymorphism rs2280073 within the GABRA4 gene and the single nucleotide polymorphism hcv2119841 within the GABRB1 gene.

15. The method of claim 3, wherein the genetic marker is a combination of the single nucleotide polymorphism rs2280073 within the GABRA4 gene and the single nucleotide polymorphism rs1862242 within the GABRP gene.

16. The method of claim 3, wherein the genetic marker is a combination of the single nucleotide polymorphism rs17599416 within the GABRA4 gene and the single nucleotide polymorphism rs2351299 within the GABRB1 gene.

17. The method of claim 3, wherein the genetic marker is a combination of the single nucleotide polymorphism rs1912960 within the GABRA4 gene, the single nucleotide polymorphism rs2351299 within the GABRB1 gene and the single nucleotide polymorphism rs7660336 within the GABRA4 gene.

18. The method of claim 3, wherein the genetic marker is a combination of the single nucleotide polymorphism rs1912960 within the GABRA4 gene, the single nucleotide polymorphism rs2351299 within the GABRB1 gene and the single nucleotide polymorphism rs17599165 within the GABRA4 gene.

19. The method of claim 3, wherein the genetic marker is a combination of the single nucleotide polymorphism rs1912960 within the GABRA4 gene, the single nucleotide polymorphism rs2351299 within the GABRB1 gene and the single nucleotide polymorphism rs17599416 within the GABRA4 gene.

20. The method of claim 3, wherein the genetic marker is a combination of the single nucleotide polymorphism rs7660336 within the GABRA4 gene, the single nucleotide polymorphism rs2351299 within the GABRB1 gene and the single nucleotide polymorphism rs17599416 within the GABRA4 gene.

21. The method of claim 3 wherein the genetic marker is a combination of the single nucleotide polymorphism RS1912960 within the GABRA4 gene, the single nucleotide polymorphism RS3114084 within the GABRB1 gene and the single nucleotide polymorphism RS2350439 within the GABRG1 gene.

22. The method of claim 3 wherein the genetic marker is a combination of the single nucleotide polymorphisms RS282117 and RS2148174 within the GABRA4 gene, and the single nucleotide polymorphism RS208129 within the GABRG3 gene.

23. A method of correlating a genetic marker within a GABAR subunit gene with an increased risk of developing an autistic disorder, comprising:
a) detecting in a subject with an autistic disorder the presence of one or more genetic markers within the GABAR subunit gene; and
b) correlating the presence of the one or more genetic markers of step (a) with the autistic disorder in the subject.

24. A method of diagnosing an autistic disorder in a subject, comprising detecting in the subject one or more genetic markers correlated with a diagnosis of an autistic disorder.

25. A method of diagnosing an autistic disorder in a subject, comprising:
a) correlating the presence of one or more genetic markers within a GABAR subunit gene with a diagnosis of an autistic disorder; and
b) detecting the one or more genetic markers of step (a) in the subject, thereby diagnosing an autistic disorder in the subject.

26. A method of correlating a genetic marker within a GABAR subunit gene with a diagnosis of an autistic disorder, comprising:
a) detecting in a subject diagnosed with an autistic disorder the presence of one or more genetic markers within the GABAR subunit gene; and
b) correlating the presence of the one or more genetic markers of step (a) with a diagnosis of an autistic disorder in a subject.

27. A method of identifying an effective treatment regimen for a subject with an autistic disorder, comprising detecting one or more genetic markers within a GABAR subunit gene in the subject correlated with an effective treatment regimen for an autistic disorder.

28. A method of identifying an effective treatment regimen for a subject with an autistic disorder, comprising:
a) correlating the presence of one or more genetic markers within a GABAR subunit gene in a test subject with an autistic disorder for whom an effective treatment regimen has been identified; and
b) detecting the one or more markers of step (a) in the subject, thereby identifying an effective treatment regimen for the subject.

29. A method of correlating a genetic marker within a GABAR subunit gene with an effective treatment regimen for autistic disorder, comprising:
a) detecting in a subject with an autistic disorder and for whom an effective treatment regimen has been identified, the presence of one or more genetic markers within a GABAR subunit gene; and
b) correlating the presence of the one or more genetic markers of step (a) with an effective treatment regimen for an autistic disorder.
